SEQUENCE LISTING (81 sequences)

SEQ ID NO: 1 - DNA, 927 bp, Arabidopsis thaliana
  CDS (1)..(927)
atgcaagtgtttcaaaggaaagaagattcatcttggggaaactcaatgcctacaacaaattcaaatattcaaggatctgaatctttcagcttgactaaggatatgataatgtctacaacacaattacccgcgatgaaacattcgggtttgcagctgcaaaatcaagattcaacctcatcacaatctactgaagaagaatcaggcggcggtgaagttgcaagctttggagaatataagcgttatggatgcagcattgttaataacaatctctcaggttacatcgaaaacttgggaaagcctattgaaaattatactaagtcaattactacctcgtcgatggtgtctcaagactctgtgtttcctgctcctacttctggtcaaatatcttggtctcttcaatgtgctgaaacgtcacatttcaatggtttcttggctcctgaatatgcatcaacaccaacggcgctgccacatttagagatgatgggtttggtttcttcaagagtgccattgcctcatcacattcaagagaatgaaccaatatttgtcaatgcgaaacagtatcatgcgattctccgtcgcaggaagcaccgtgctaaactcgaagctcagaacaaactcatcaaatgccgtaaaccgtaccttcatgagtctcgccatcttcatgctttaaagagagctagaggctccggtggacgtttcctcaatacaaagaagcttcaagaatcatcaaactcactgtgttcttctcaaatggcaaatggacaaaatttctctatgagccctcacggtggtggaagcggaatcgggtctagttcgatctcaccgagctccaattcaaactgtatcaacatgttccaaaacccgcagttcagattctcaggttatccgtcaacacaccatgcctcagctctcatgtcagggacttga

SEQ ID NO: 2 - protein, 308 aa, Arabidopsis thaliana
MQVFQRKEDSSWGNSMPTTNSNIQGSESFSLTKDMIMSTTQLPAMKHSGLQLQNQDSTSSQSTEEESGGGEVASFGEYKRYGCSIVNNNLSGYIENLGKPIENYTKSITTSSMVSQDSVFPAPTSGQISWSLQCAETSHFNGFLAPEYASTPTALPHLEMMGLVSSRVPLPHHIQENEPIFVNAKQYHAILRRRKHRAKLEAQNKLIKCRKPYLHESRHLHALKRARGSGGRFLNTKKLQESSNSLCSSQMANGQNFSMSPHGGGSGIGSSSISPSSNSNCINMFQNPQFRFSGYPSTHHASALMSGT

SEQ ID NO: 3 - DNA, 1023 bp, Oryza sativa
  CDS (1)..(1023)
atgtcggcacccttcatccaccaagccgctgcccacaggctacctcttattcgagaaaaagaaaaagaggctgataagtacacgaagaaaaatggtgcctggaccaaagtgagaccatctcataatgctaatgcttttgcgacaaatggaagatcatcgagcccatactacgcccaactatattatcagatgaagcagttaggtcataagatgtatgatcaagattccccgtcaagcgattctggacagtcacaccaagaagaatctgccatgaatgatagcagtccaaatgagcgacatacctcaacacaatctgacaatgatgatggtcatcagatgccagatcaggacaaaacaaagtcagtatcatcgttggggaatccaggagctttgcctccaaagctcaactatagccaatcctttgcttgtattccttatacagctgatgcatactatggtggggtcttgactggatattcttcacatgcaattgttcatccccagcaaaatggtacagctaactcccgggtgccgttgcctgttgagcctgcagcagaagagccaatatttgtaaacgcaaagcagtaccatgcaattcttaggaggagacagatacgagctaaattggaggcccaaaataagttggtgaaaggtcggaagccatacctccatgaatctcggcatcgccatgccatgaagcgagctcgtggatctggagggcgcttcctcaacacaaagcagctcgaggagcagaagcagcaacaggaggaggaggccgcatccggtggcgcgagctctggcaataggacatgccttcagaatggcaccggtagcgcgccttcagcttcctctccctctgaaatcgcgagcgtctcaaccagcagagaattccttggtaaccatgagcagagccacttcccctcagctggcttccttcccacaatgagcttcagagcgcagaatggcggagatgggaagctggtcgcgaacgccattcaccagcgcgtttccatgatgaggtga

SEQ ID NO: 4 - protein, 340 aa, Oryza sativa
MSAPFIHQAAAHRLPLIREKEKEADKYTKKNGAWTKVRPSHNANAFATNGRSSSPYYAQLYYQMKQLGHKMYDQDSPSSDSGQSHQEESAMNDSSPNERHTSTQSDNDDGHQMPDQDKTKSVSSLGNPGALPPKLNYSQSFACIPYTADAYYGGVLTGYSSHAIVHPQQNGTANSRVPLPVEPAAEEPIFVNAKQYHAILRRRQIRAKLEAQNKLVKGRKPYLHESRHRHAMKRARGSGGRFLNTKQLEEQKQQQEEEAASGGASSGNRTCLQNGTGSAPSASSPSEIASVSTSREFLGNHEQSHFPSAGFLPTMSFRAQNGGDGKLVANAIHQRVSMMR

SEQ ID NO: 5 - DNA, 834 bp, Oryza sativa
  CDS (1)..(834)
atgaagcagttaggtcataagatgtatgatcaagattccccgtcaagcgattctggacagtcacaccaagaagaatctgccatgaatgatagcagtccaaatgagcgacatacctcaacacaatctgacaataatgatggtcatcagatgccagatcaggacaaaacaaagtcagtatcatcgttggggaatccaggagctttgcctccaaagctcgactatagccaatcctttgcttgtattccttatacagctgatgcatactatggtggggtcttgactggatattcttcacatgcaattgttcatccccagcaaaatggtacagctaactcccgggtgccgttgcctgttgagcctgcagcagaagagccaatatttgttaacgcaaagcagtaccatgcaattcttaggaggagacagatacgagctaaattggaggcccaaaataagttggtgaaaggtcggaagccatacctccatgaatctcggcatcgccatgccatgaagcgagctcgtggatctggagggcgcttcctcaacacaaagcagctcgaggagcagaagcagcaacaggaggaggaggccgcatccggtggcgcgagctctggcaataggacatgccttcagaatggcaccggtagcgcgccttcagcttcctctccctctgaaatcgcgagcgtctcaaccagcagagaattccttggtaaccatgagcagagccacttcccctcagctggcttccttcccacaatgagcttcagagcgcagaatggcggagatgggaagctggtcgcgaacgccattcaccagcgcgtttccatgatgaggtga

SEQ ID NO: 6 - protein, 277 aa, Oryza sativa
MKQLGHKMYDQDSPSSDSGQSHQEESAMNDSSPNERHTSTQSDNNDGHQMPDQDKTKSVSSLGNPGALPPKLDYSQSFACIPYTADAYYGGVLTGYSSHAIVHPQQNGTANSRVPLPVEPAAEEPIFVNAKQYHAILRRRQIRAKLEAQNKLVKGRKPYLHESRHRHAMKRARGSGGRFLNTKQLEEQKQQQEEEAASGGASSGNRTCLQNGTGSAPSASSPSEIASVSTSREFLGNHEQSHFPSAGFLPTMSFRAQNGGDGKLVANAIHQRVSMMR

SEQ ID NO: 7 - DNA, 729 bp, Triticum aestivum
  CDS (1)..(729)
atgagacgcaagcaaaatatgcatgaaaatggcacaatcatgattcagtttggtcagcaagtgcctaactgcgagtcctcagctagcgattctcctcaagaggtgtccggaatgagcgaaggaagctttaatgagcagaatgatcaatctggtaaccgcgatggctacacgaagagtgatgaaggcaagatgatgtcagctttgtctctgggcaattcagaaacagcatacacaccgccaaaacctgaccgcactcatccctttgccatatcatacccatatgctgatccttactatggtggtgcagtggcagcctatggcgcacatgctattatgcacccccagatggtgggcatggtaccatcctctcgagtgccgctgccaattgaaccggccgccgccgaagagcccatttatgtgaacgcgaagcaataccatgccattctccgaaggagacagctccgcgcaaaattagaggctgaaaataagctggtgaagagccgcaagccgtacctccacgagtcccggcaccagcacgcgatgaagcgggcccggggaacaggcgggcggttcctcaacgcgaaggagaagtccgaggcctccggcggcggcaatgcatcagcgagacctggctacgccggcgttcctgcagacggcggcatgttctcgaagcacgaccacaccttaccgtccggtgacttccattaccgcgcgagagggggcgcctag

SEQ ID NO: 8 - protein, 242 aa, Triticum aestivum
MRRKQNMHENGTIMIQFGQQVPNCESSASDSPQEVSGMSEGSFNEQNDQSGNRDGYTKSDEGKMMSALSLGNSETAYTPPKPDRTHPFAISYPYADPYYGGAVAAYGAHAIMHPQMVGMVPSSRVPLPIEPAAAEEPIYVNAKQYHAILRRRQLRAKLEAENKLVKSRKPYLHESRHQHAMKRARGTGGRFLNAKEKSEASGGGNASARPGYAGVPADGGMFSKHDHTLPSGDFHYRARGGA

SEQ ID NO: 9 - DNA, 26 bp, Artificial Sequence
  Primer sequence
caccatgcaagtctttcaaaggaaag

SEQ ID NO: 10 - DNA, 22 bp, Artificial Sequence
  Primer Sequence
gtaatgcaattgtactctcgag

SEQ ID NO: 11 - DNA, 26 bp, Artificial Sequence
  Primer sequence
tcaagtccctgacatgagagctgagg

SEQ ID NO: 12 - DNA, 28 bp, Artificial Sequence
  Primer sequence
caccatgtcctcgccggagcgtgctctc

SEQ ID NO: 13 - DNA, 23 bp, Artificial Sequence
  Primer Sequence
tagttcgatctcaccgagctcca

SEQ ID NO: 14 - DNA, 26 bp, Artificial Sequence
  Primer sequence
cacctgggtatagctagtgaaacgcg

SEQ ID NO: 15 - DNA, 21 bp, Artificial Sequence
  Primer sequence
ccttagcttgagttcttgcga

SEQ ID NO: 16 - DNA, 22 bp, Artificial Sequence
  Primer sequence
cacccccaacggagtagaattg

SEQ ID NO: 17 - DNA, 23 bp, Artificial Sequence
  Primer sequence
ctcatacggtcgatgtaatccgt

SEQ ID NO: 18 - DNA, 22 bp, Artificial Sequence
  Primer sequence
cacctcgtccattatgagtatt

SEQ ID NO: 19 - DNA, 24 bp, Artificial Sequence
  Primer sequence
ctaatatgatatgaatatggatga

SEQ ID NO: 20 - DNA, 24 bp, Artificial Sequence
  Primer sequence
cacctcatataagagaaaatggtg

SEQ ID NO: 21 - DNA, 24 bp, Artificial Sequence
  Primer sequence
ccaaaaaagagaaatgtgaatgag

SEQ ID NO: 22 - DNA, 27 bp, Artificial Sequence
  Primer sequence
cacctgtatgacatattctgtgtggag

SEQ ID NO: 23 - DNA, 22 bp, Artificial Sequence
  Primer sequence
tgcaaattgggtattggctatg

SEQ ID NO: 24 - DNA, 22 bp, Artificial Sequence
  Primer sequence
tgggtatagctagtgaaacgcg

SEQ ID NO: 25 - DNA, 21 bp, Artificial Sequence
  Primer sequence
ccttagcttgagttcttgcga

SEQ ID NO: 26 - DNA, 24 bp, Artificial Sequence
  Primer sequence
ctccactccctaaaccatcacaac

SEQ ID NO: 27 - DNA, 24 bp, Artificial Sequence
  Primer sequence
ctcatacggtcgatgttaatccgt

SEQ ID NO: 28 - DNA, 23 bp, Artificial Sequence
  Primer sequence
ctacattcacaaacgagagaatt

SEQ ID NO: 29 - DNA, 23 bp, Artificial Sequence
  Primer sequence
gctctactttagcatctcaacca

SEQ ID NO: 30 - DNA, 23 bp, Artificial Sequence
  Primer sequence
gtagtcttgtgggatactcatca

SEQ ID NO: 31 - DNA, 24 bp, Artificial Sequence
  Primer sequence
atccccaaatttggaggccaaaca

SEQ ID NO: 32 - DNA, 23 bp, Artificial Sequence
  Primer sequence
aaggtgtctcctgggttgaaaat

SEQ ID NO: 33 - DNA, 23 bp, Artificial Sequence
  Primer sequence
ccatgatcattcatgtcgtgcct

SEQ ID NO: 34 - DNA, 22 bp, Artificial Sequence
  Primer sequence
gagagcacatccatgtgaggaa

SEQ ID NO: 35 - DNA, 22 bp, Artificial Sequence
  Primer sequence
gtcaggcaagtcatccttggct

SEQ ID NO: 36 - DNA, 23 bp, Artificial Sequence
  Primer sequence
ctctttggctatcttgacatgct

SEQ ID NO: 37 - DNA, 22 bp, Artificial Sequence
  Primer sequence
ccaaggagactgcctgatgtct

SEQ ID NO: 38 - DNA, 22 bp, Artificial Sequence
  Primer sequence
gagagcacatgaatgtaaggca

SEQ ID NO: 39 - DNA, 24 bp, Artificial Sequence
  Primer sequence
aataatgtgtagactcagccacat

SEQ ID NO: 40 - DNA, 22 bp, Artificial Sequence
  Primer sequence
cttgcgggttaggtttcaggca

SEQ ID NO: 41 - DNA, 24 bp, Artificial Sequence
  Primer sequence
gcctcgaaatcatgaacattatct

SEQ ID NO: 42 - DNA, 22 bp, Artificial Sequence
  Primer sequence
agagagcacatgcatgtatgga

SEQ ID NO: 43 - DNA, 24 bp, Artificial Sequence
  Primer sequence
gaaaagtaggtataacatggatgg

SEQ ID NO: 44 - DNA, 23 bp, Artificial Sequence
  Primer sequence
gaagattcatcttggggaaactc

SEQ ID NO: 45 - DNA, 23 bp, Artificial Sequence
  Primer sequence
gagcaggaaacacagagtcttga

SEQ ID NO: 46 - DNA, 24 bp, Artificial Sequence
  Primer sequence
ggttgggtgtttcccggcatcgga

SEQ ID NO: 47 - DNA, 25 bp, Artificial Sequence
  Primer sequence
ggcttctcctaagatctgatctcca

SEQ ID NO: 48 - DNA, 23 bp, Artificial Sequence
  Primer sequence
ccttccctccgatccttacaaga

SEQ ID NO: 49 - DNA, 25 bp, Artificial Sequence
  Primer sequence
caacccaagtttcttcctacgttcg

SEQ ID NO: 50 - DNA, 22 bp, Artificial Sequence
  Primer sequence
cgttgaggcggcaggtgagtgt

SEQ ID NO: 51 - DNA, 23 bp, Artificial Sequence
  Primer sequence
tgtaacgtccacatcgcttgcca

SEQ ID NO: 52 - DNA, 24 bp, Artificial Sequence
  Primer sequence
tcagtggcatgggttcttcttcca

SEQ ID NO: 53 - DNA, 23 bp, Artificial Sequence
  Primer sequence
gaggtcatcgaggatgttgccgt

SEQ ID NO: 54 - DNA, 22 bp, Artificial Sequence
  Primer sequence
gttctcactggtatggcttctt

SEQ ID NO: 55 - DNA, 25 bp, Artificial Sequence
  Primer sequence
gtctttcgctttctcaccatctgct

SEQ ID NO: 56 - DNA, 19 bp, Artificial Sequence
  Primer sequence
aggaattgacggaagggca

SEQ ID NO: 57 - DNA, 21 bp, Artificial Sequence
  Primer sequence
gtgcggcccagaacatctaag

SEQ ID NO: 58 - DNA, 25 bp, Artificial Sequence
  Primer sequence
ataaccgtttcaaattctctctctc

SEQ ID NO: 59 - DNA, 20 bp, Artificial Sequence
  Primer sequence
tgcaaatcgttctctccttg

SEQ ID NO: 60 - DNA, 13 bp, Artificial Sequence
  Consensus sequence
tnnttngnncant

SEQ ID NO: 61 - DNA, 21 bp, Arabidopsis thaliana
cagccaaggatgacttgccga

SEQ ID NO: 62 - DNA, 136 bp, Arabidopsis thaliana
attcataagcaccaaaacaaatatgtagagatctcctcttccattctctattgttactttcgagaagaaacatacaaaacacatacatttttcttttgtttgtggttttcatatatacaagtgggtatagctagtg

SEQ ID NO: 63 - protein, 56 aa, Arabidopsis sp.
VNAKQFHAIMRRRQQRAKLEAQNKLIKARKPYLHESRHVHALKRPRGSGGRFLNTK

SEQ ID NO: 64 - protein, 56 aa, Arabidopsis sp.
VNAKQYHAILRRRKHRAKLEAQNKLIKCRKPYLHESRHLHALKRARGSGGRFLNTK

SEQ ID NO: 65 - protein, 56 aa, Arabidopsis sp.
VNAKQYHAIMRRRQQRAKLEAQNKLIRARKPYLHESRHVHALKRPRGSGGRFLNTK

SEQ ID NO: 66 - protein, 56 aa, Arabidopsis sp.
VNAKQYHGILRRRQSRAKLEARNRAIKAKKPYMHESRHLHAIRRPRGCGGRFLNAK

SEQ ID NO: 67 - protein, 60 aa, Arabidopsis sp.
VNSKQYHGIIRRRQSRAKAAAVLDQKKLSSRCRKPYMHHSRHLHALRRPRGSGGRFLNTK

SEQ ID NO: 68 - protein, 56 aa, Arabidopsis sp.
VNAKQYQAILRRRERRAKLEAQNKLIKVRKPYLHESRHLHALKRVRGSGGRFLNTK

SEQ ID NO: 69 - protein, 56 aa, Arabidopsis sp.
VNAKQYQAILRRRQARAKAELEKKLIKSRKPYLHESRHQHAMRRPRGTGGRFAKKT

SEQ ID NO: 70 - protein, 52 aa, Arabidopsis sp.
VNSKQYHGIIRRQSRAKAEKLSRCRKPYMHHSRHLHAMRRPRGSGGRFLNTK

SEQ ID NO: 71 - protein, 56 aa, Arabidopsis sp.
VNAKQYEGILRRRKARAKAELERKVIRDRKPYLHESRHKHAMRRARASGGRFAKKS

SEQ ID NO: 72 - DNA, 1071 bp, Oryza sativa
  CDS (1)..(1071)
atgtcggcacccttcatccaccaagccgctgcccacaggctacctcttattcgagaaaaagaaaaagaggctgataagtacacgaagaaaaatggtgcctggaccaaagtgagaccatctcataatgctaatgcttttgcgacaaatggaagatcatcgagcccatactacgcccaactatattatcagatgaagcagttaggtcataagatgtatgatcaagattccccgtcaagcgattctggacagtcacaccaagaagaatctgccatgaatgatagcagtccaaatgagcgacatacctcaacacaatctgacaatgatgatggtcatcagatgccagatcaggacaaaacaaagtcagtatcatcgttggggaatccaggagcttatcaggacaaaacaaagtcagtatcatcgttggggaatccaggagctttgcctccaaagctcaactatagccaatcctttgcttgtattccttatacagctgatgcatactatggtggggtcttgactggatattcttcacatgcaattgttcatccccagcaaaatggtacagctaactcccgggtgccgttgcctgttgagcctgcagcagaagagccaatatttgtaaacgcaaagcagtaccatgcaattcttaggaggagacagatacgagctaaattggaggcccaaaataagttggtgaaaggtcggaagccatacctccatgaatctcggcatcgccatgccatgaagcgagctcgtggatctggagggcgcttcctcaacacaaagcagctcgaggagcagaagcagcaacaggaggaggaggccgcatccggtggcgcgagctctggcaataggacatgccttcagaatggcaccggtagcgcgccttcagcttcctctccctctgaaatcgcgagcgtctcaaccagcagagaattccttggtaaccatgagcagagccacttcccctcagctggcttccttcccacaatgagcttcagagcgcagaatggcggagatgggaagctggtcgcgaacgccattcaccagcgcgtttccatgatgaggtga

SEQ ID NO: 73 - protein, 356 aa, Oryza sativa
MSAPFIHQAAAHRLPLIREKEKEADKYTKKNGAWTKVRPSHNANAFATNGRSSSPYYAQLYYQMKQLGHKMYDQDSPSSDSGQSHQEESAMNDSSPNERHTSTQSDNDDGHQMPDQDKTKSVSSLGNPGAYQDKTKSVSSLGNPGALPPKLNYSQSFACIPYTADAYYGGVLTGYSSHAIVHPQQNGTANSRVPLPVEPAAEEPIFVNAKQYHAILRRRQIRAKLEAQNKLVKGRKPYLHESRHRHAMKRARGSGGRFLNTKQLEEQKQQQEEEAASGGASSGNRTCLQNGTGSAPSASSPSEIASVSTSREFLGNHEQSHFPSAGFLPTMSFRAQNGGDGKLVANAIHQRVSMMR

SEQ ID NO: 74 - DNA, 834 bp, Oryza sativa; identical to SEQ ID NO: 5
  CDS (1)..(834)

SEQ ID NO: 75 - protein, 277 aa, Oryza sativa; identical to SEQ ID NO: 6

SEQ ID NO: 76 - DNA, 729 bp, Triticum aestivum; identical to SEQ ID NO: 7
  CDS (1)..(729)

SEQ ID NO: 77 - protein, 242 aa, Triticum aestivum; identical to SEQ ID NO: 8

SEQ ID NO: 78 - DNA, 978 bp, Arabidopsis thaliana
  CDS (1)..(978)
atgaatgtgcaagtgtttcaaaggaaagaagattcatcttggggaaactcaatgcctacaacaaattatgcaaatattcaaggatctgaatctttcagcttgactaaggatatgataatgtctacaacacatgaattacccgcgatgaaacattcgggtttgcagctgcaaaatcaagattcaacctcatcacatgaatctactgaagaagaatcaggcggcggtgaagttgcaagctttggagaatataagcgttatgatggatgcagcattgttaataacaatctctcaggttacatcgaaaacttgggaaagcctaatgttgaaaattatactaagtcaattactacctcgtcgatggtgtctcaagactctgatgtgtttcctgctcctacttctggtcaaatatcttggtctcttcaatgtgctgaaacgtcaatgcatttcaatggtttcttggctcctgaatatgcatcaacaccaacggcgctgccacatttaatggagatgatgggtttggtttcttcaagagtgccattgcctcatcacattcaagagaatatggaaccaatatttgtcaatgcgaaacagtatcatgcgattctccgtcgcaggaagcaccgtatggctaaactcgaagctcagaacaaactcatcaaatgccgtaaaccgtaccttcatgagtctatgcgccatcttcatgctttaaagagagctagaggctccggtggacgtttcctcaatacaaagatgaagcttcaagaatcatcaaactcactgtgttcttctcaaatggcaaatggacaaaattatgtctctatgagccctcacggtggtggaagcggaatcgggtctagttcgatctcaccgagatgctccaattcaaactgtatcaacatgttccaaaacccgcagttcagattctcaggttatcatgcgtcaacacaccatgcctcagctctcatgtcagggacttga

SEQ ID NO: 79 - protein, 325 aa, Arabidopsis thaliana
MNVQVFQRKEDSSWGNSMPTTNYANIQGSESFSLTKDMIMSTTHELPAMKHSGLQLQNQDSTSSHESTEEESGGGEVASFGEYKRYDGCSIVNNNLSGYIENLGKPNVENYTKSITTSSMVSQDSDVFPAPTSGQISWSLQCAETSMHFNGFLAPEYASTPTALPHLMEMMGLVSSRVPLPHHIQENMEPIFVNAKQYHAILRRRKHRMAKLEAQNKLIKCRKPYLHESMRHLHALKRARGSGGRFLNTKMKLQESSNSLCSSQMANGQNYVSMSPHGGGSGIGSSSISPRCSNSNCINMFQNPQFRFSGYHASTHHASALMSGT

SEQ ID NO: 80 - DNA, 19 bp, Arabidopsis thaliana
tagccaaggatgacttccc

SEQ ID NO: 81 - DNA, 19 bp, Arabidopsis thaliana
atcaauuaauacugaaggg

(19) United States
(12) Patent Application Publication
(10) Pub. No.: US 2011/0207608 A1
(43) Pub. Date: Aug. 25, 2011

(54) TRANSCRIPTIONAL AND POST-TRANSCRIPTION REGULATION OF TRANSCRIPTION FACTOR FOR DROUGHT RESISTANCE

(75) Inventors: Jian-Kang Zhu, Newport Coast, CA (US); Wen-Xue Li, Beijing (CN)

(73) Assignee: THE REGENTS OF THE UNIVERSITY OF CALIFORNIA, Oakland, CA (US)

(21) Appl. No.: 12/990,211

(22) PCT Filed: Apr. 30, 2009
(86) PCT No.: PCT/US09/42430

§ 371 Date: May 10, 2011

Related U.S. Application Data
(60) Provisional application No. 61/048,996, filed on Apr. 30, 2008.

(51) Int. Cl. A01N 25/00; C07H 21/04; C12N 15/63; C12N 5/10; A01H 5/00; A01H 1/00; C12Q 1/68

(52) U.S. Cl. 504/116.1; 536/23.6; 435/320.1; 435/419; 800/298; 800/278; 435/6.13

(57) ABSTRACT

The disclosure provides polynucleotides useful in generating drought resistant plants, transgenic plants comprising modified expression of such a polynucleotide and progeny thereof. Also provided are molecules useful in providing drought resistance to a plant.

### CROSS REFERENCE TO RELATED APPLICATIONS

This application claims priority to U.S. Provisional Application No. 61/048,996, filed Apr. 30, 2008, the disclosure of which is incorporated herein by reference.

### STATEMENT WITH REGARD TO FEDERAL SPONSORED RESEARCH

This invention was supported by a grant from the National Institutes of Health, Grant No. R01GM059138. The government has certain rights in this invention.

### BACKGROUND

Drought stress is a major environmental factor limiting crop productivity worldwide. To reduce the adverse effects of drought stress, plants have evolved multifaceted strategies, including morphological, physiological and biochemical adaptations (Bohnert et al., 2006; Shinozaki et al., 2003; Xiong et al., 2002; Zhu, 2002; Ingram and Bartels, 1996). Some of these strategies aim to avoid dehydration stress by increasing water uptake or reducing water loss while other strategies seek to protect plant cells from damage when water is depleted and tissue dehydration becomes inevitable (Verslues et al., 2006). Changes in gene expression play an important role in plant drought stress response and many stress induced genes are known or presumed to have roles in drought resistance. For many of these genes, the hormone abscisic acid (ABA) is a key signaling intermediate controlling their expression. This has been shown in large part by analysis of ABA-deficient and ABA-insensitive mutants in Arabidopsis (Koomeef et al., 1998).

Numerous environmental factors effect a plant's growth and fruiting, with soil salinity and drought having the most detrimental effects. Approximately 70% of the genetic yield potential in major crops is lost due to abiotic stresses, and most major agricultural crops are susceptible to drought stress. Attempts to improve yield under stress conditions by plant breeding have been largely unsuccessful, primarily due to the multigenic origin of the adaptive responses (Barkla et al. 1999, Adv Exp Med Biol 464:77-89).

### SUMMARY

The disclosure demonstrates that overexpression of NFYA5 improves drought resistance. The disclosure provides that part of the role of NFYA5 in drought resistance involves its expression in guard cells and control of stomatal aperture. In addition, NFYA5 is broadly expressed in various tissues. In non-guard cells, NFYA5 is likely important for dehydration tolerance via its role in activating target stress-responsive genes such as genes involved in oxidative stress responses. The candidate target genes of AtNFYB1 do not have obvious associations with stress tolerance and some of them appear to be related to polysaccharide metabolism. The lack of substantial overlap between the target genes of NFYA5 and AtNFYB1 indicates that the two transcription factors may be involved in separate gene regulons.

Transcriptional induction explain part of NFYA5 transcript accumulation under drought stress. ABA is involved in the transcriptional regulation since ABA causes NFYA5 transcript accumulation and it activates NFYA5 promoter activity.

A feature of NFYA5 regulation under drought stress is the involvement of a miRNA. The data presented herein demonstrate that the accumulation of NFYA5 transcript is suppressed by miR169. Drought stress down-regulates miR169 expression, thus relieving miR169 repression of NFYA5. miR169 is encoded by many loci. Only two of the loci, MIR169a and MIR169c, are substantially down-regulated by drought stress. The disclosure indicates that miR169a rather than miR169c plays a major role in repressing NFYA5 transcript accumulation, despite the fact that both miRNAs have three mismatches with NFYA mRNA. ABA is required for the downregulation of MIR169a and MIR169c by drought stress. Therefore, ABA is involved in both the transcriptional and posttranscriptional regulation of NFYA5. The downregulation of MIR169a and MIR169c by ABA and drought stress likely involves transcriptional repression at the two loci.

NFYA5 is regulated by drought stress not only transcriptionally but also posttranscriptionally via a miRNA. This dual level regulation is consistent with the importance of NFYA5 for drought resistance. Both NFYA5 and miR169 are highly conserved in rice (Jones-Rhoades and Bartel, 2004), so the dual modes of regulation of NFYA5 also apply to other plants.

The disclosure provides an isolated polynucleotide comprising: an NFY5A polynucleotide, homolog or otholog thereof operably linked to a heterologous promoter. In one embodiment, the NFY5A comprises at least 50%, 60%, 70%, 80%, 90%, 95%, 98%, 99%, 100% identity to a polynucleotide consisting of SEQ ID NO:1, 3, 5, or 7. In another embodiment, the NFY5A polynucleotide encodes a polypeptide comprising SEQ ID NO:2. In yet another embodiment, the heterologous promoter comprises a constitutive promoter or an inducible promoter. In one aspect, the constitutive promoter is a cauliflower mosaic virus 35S or 19S promoter or a plant ACT2 promoter or a plant ubiquitin promoter. In another aspect, the constitutive promoter is an Actin2 promoter derived from Arabidopsis thaliana. In yet another aspect, the inducible promoter comprises a light inducible promoter.

The disclosure also provides a plant cell transformed with a polynucleotide as set forth above.

The disclosure also provides a transgenic plant that expresses an NFY5A polynucleotide at a level higher than that of a wild-type plant and the transgenic plant has improved drought tolerance.

The disclosure further provides a transgenic plant that lacks the 3′UTR of a NFY5A gene, homolog or ortholog thereof.

The disclosure provides a transgenic plant that lacks a functional miR169 polynucleotide in the genome. For example, the transgenic plant can lack a functional miR169a or c polynucleotide in the genome.

The disclosure also provides a method of using the polynucleotide as described above to produce a plant which is resistant to drought, comprising the steps of introducing the polynucleotide or construct into a plant cell or into plant tissue, selecting for the presence of the polynucleotide molecule to produce a transgenic plant cell or transgenic plant tissue, and regenerating a plant from the transgenic plant cell or transgenic plant tissue, whereby a drought resistant plant is generated. In one embodiment, the polynucleotide comprises a sequence encoding an NFY5A polypeptide operably linked to a heterolgous promoter. In another embodiment, the polynucleotide comprises a construct that reduces the expression of an mir169a or c or which provides a complement of an mir169a or c or which knocks out mir169a or c in a plant cell.

The disclosure also provides a method for identifying agents useful for modifying drought resistance in a plant comprising: contacting a plant that contains an NFY5A gene with an agent; measuring a change in expression of the gene or the amount of mRNA transcript present in the cell; wherein a change in expression or transcript levels compared to a control is indicative of an agent that modifies drought resistance.

The disclosure provides a method for identifying agents useful for modifying drought resistance in a plant comprising: contacting a plant with an agent, the lant comprising a sequence of TN(C/A)TTNGN(C/A)CANT (SEQ ID NO: 60) operably linked to a reporter gene; measuring a change in expression of the reporter gene; wherein a change in the expression or the reporter gene compared to a control is indicative of an agent that modifies drought resistance.

The disclosure provides a method of increasing drought tolerance in a plant comprising contact a plant with an agent the increases the expression of (1) an NFY5A gene, homolog or ortholog thereof and/or (2) a gene listed in Table 1.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

FIG. 1A-B shows regulation of NFYA5 expression by dehydration and ABA. (a) Assay of the accumulation of NFYA5 gene transcripts in Arabidopsis seedlings in response to dehydration and ABA by real-time RT-PCR. The expression levels were normalized to that of Tub8. Error bars are SD of three replicates. (b) Detection of NFYA5 mRNA in ABA deficient or signaling mutants. Wild-type (WT) and mutants were grown with sufficient water for 3 weeks, and then water was withheld for 10 days. Twenty μg of total RNA from each sample was loaded and hybridized with ³²P-labeled full length NFYA5 cDNA probe. The Tub8 was used as a loading control and numbers below each lane indicate the expression level relative to Tub8.

FIG. 2A-D shows NFYA5 expression pattern and transcriptional regulation. (a) Two week-old transgenic seedlings on MS-agar medium were exposed to either 3 h dehydration or 8 h ABA treatment and then stained for GUS activity. (b)NFYA5p::GUS expression pattern in various tissues. The staining was prominent in the vascular tissues (I) and guard cells (II) of leaves. The staining was also visible in floral tissues of inflorescence (III) and root vascular system (IV). (c) Tissue pattern of NFYA5 transcript accumulation. Total RNA was isolated from various tissues of 4-week-old wild-type plants grown under long-day growth conditions. Real-time RT-PCR quantifications were normalized to the expression of 18S rRNA. The results represent SD of three replicates. (d) Subcellular localization of NFYA5. NFYA5-YFP fusion construct was expressed in transgenic Arabidopsis under the control of CaMV 35S promoter and the plant roots were observed under a confocal microscope. The photographs were taken in the dark field for yellow fluorescence (I), in the bright field for the morphology of the cells (II) and in combination (III).

FIG. 3A-D shows drought stress down-regulates a 21 nt small RNA that is complementary to NFYA5 mRNA. (a) Scheme of the cis-antisense gene pair of NFYA5 and At1g54150. Exons are boxed and lines between boxes represent introns. Arrow indicates target position of the small RNA. (b) Regulation of the small RNA by drought stress and ABA treatment. U6 RNA was probed for loading controls. Numbers below each lane indicate the relative expression ratio. (c) Accumulation of the small RNA in various RNA silencing mutants. Forty micrograms of small RNA from each sample was loaded per lane and hybridized with ³²P-labeled oligonucleotide probe corresponding to the sequence of ASRP1815. miR172, siR255 and U6 were shown as loading controls. (d) NFYA5 mRNA levels in dcl1-7, hen1 and hyl1 and their corresponding wild types. The expression levels were normalized to that of Tub8. The results represent SD of three replicates.

FIG. 4A-E shows NFYA5 is mainly regulated by miR169a. (a) Detection of precursor transcripts of MIR169 family in response to drought stress by real-time RT-PCR. Quantifications were normalized to the expression of Tub8. The results represent SD of three replicates. (b) Diagram of NFYA5 expression constructs. The introduced mutations in the target site of NFYA5 were shown in lowercase letters (SEQ ID NOs: 80 and 81). Black box in the 3′ UTR indicates the miRNA target site. (c) Coexpression of various combinations of miR169 and NFYA5 expression constructs in N. benthamiana. As a control, NFYA5 was also coexpressed with an unrelated YFP construct. Real-time RT-PCR quantifications were normalized to the expression of 18S rRNA of tobacco. The results represent SD of three replicates. (d) Overexpression of miR169a and miR169c in transgenic Arabidopsis. Northern blot analysis of miR169a and miR169c levels in wild type and two representative transgenic lines. miR171 was shown as a loading control. Numbers below each lane indicate the relative expression ratio. (e) Detection of corresponding NFYA5 gene transcripts in 35S:MIR169 transgenic plant lines by real-time RT-PCR. Quantifications were normalized to the expression of Tub 8. The results represent SD of three replicates.

FIG. 5A-D shows 35S::MIR169a plants are more sensitive to drought stress. (a) 35S::MIR169a overexpressing Arabidopsis plants are more sensitive to drought stress. Wild type (Col) and 35S::MIR169a plants were grown in soil with sufficient water for 3 weeks, and then the water was withheld for 8 days. A representative picture is shown. Control, without water withholding. (b) Measurement of stomatal aperture in wild type and 35S::MIR169a plants. Data are mean ratios of width to length±SE of three independent experiments (n=40-50). (c) Water loss from detached leaves of wild type and 35S::MIR169a plants. Water loss was expressed as the percentage of initial fresh weight. Values are means from 10 leaves for each of four independent experiments. (d) Anthocyanin content in leaves of Arabidopsis with or without drought treatment for 8 days. The results represent SD of four replicates.

FIG. 6A-E shows nfya5 mutant plants are more sensitive to drought stress. (a) Schematic diagram of the T-DNA insertion site in the NFYA5 locus and detection of NFYA5 mRNA by Northern blot analysis. Exons are boxed and lines between boxes represent introns. Twenty micrograms of total RNA from each sample was loaded and hybridized with ³²P-labeled full length NFYA5 probe. The corresponding ethidium bromide-stained rRNA is shown as a loading control. (b) nfya5 mutant plants are more sensitive to drought stress. Wild type (Col) and nfya5 plants were grown in soil with sufficient water for 3 weeks, and then the water was withheld for 8 days. A representative picture is shown. (c) Measurement of stomatal aperture in wild type and nfya5 mutant plants. Data are mean ratios of width to length±SE of three independent experiments (n=40-50). (d) Water loss from detached leaves of wild type and nfya5 mutant plants. Water loss was expressed as the percentage of initial fresh weight. Values are means from 10 leaves for each of four independent experiments. (e) Anthocyanin content in leaves of Arabidopsis with or without drought treatment for 8 days. The results represent SD of four replicates.

FIG. 7A-E shows improved drought resistance in 35S::NFYA5 plants. (a) Detection of NFYA5 mRNA in 35S::NFYA5 transgenic Arabidopsis. Real-time RT-PCR quantifications were normalized to the expression of Tub8. The results represent SD of three replicates. (b) Drought resistance of 35S::NFYA5 plants (lines 2, 3 and 5). Wild type and 35S::NFYA5 Arabidopsis plants were grown in soil with sufficient water for 3 weeks, and then the water was withheld for 14 days. A representative picture is shown. (c) Measurement of stomatal aperture in wild type and 35S::NFYA5-3 transgenic plants. Data are mean ratios of width to length±SE of three independent experiments (n=40-50). (d) Water loss from detached leaves of wild type and 35S::NFYA5-3 plants. Water loss was expressed as the percentage of initial fresh weight. Values are means from 10 leaves for each of four independent experiments. (e) Anthocyanin content in leaves of Arabidopsis with or without drought treatment for 14 days. The results represent SD of four replicates.

FIG. 8 shows a sequence alignment of the conserved domains in Arabidopsis NFYA family members (going down—SEQ ID NOs: 63-71, respectively).

FIG. 9 shows an analysis of miR169 and NFYA5 mRNA levels in 35S:MIR169b and 35S:MIR169h transgenic plant lines. Real-time RT-PCR quantification of NFYA5 transcript levels was normalized to the expression of Tub 8. The results represent SD of three replicates.

FIG. 10 shows an analysis of transcript levels in the wild-type and 35S::NFYA5 transgenic plants. Real-time RT-PCR was used for analyzing the expression levels of indicated loci. Error bars indicate SD (n=3).

FIG. 11A-B show DNA alignments for NFYA5 from arabidopsis, wheat, and two strains of rice (Os_J (SEQ ID NO:72); Os_I (SEQ ID NO:74); Wheat (SEQ ID NO:76); Arabidopsis (SEQ ID NO:78)).

FIG. 12 shows sequences of the disclosure (SEQ ID NOs: 1, 2, 61 and 62).

### DETAILED DESCRIPTION

As used herein and in the appended claims, the singular forms “a,” “and,” and “the” include plural referents unless the context clearly dictates otherwise. Thus, for example, reference to “a polynucleotide” includes a plurality of such polynucleotides and reference to “the peptide” includes reference to one or more peptides, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of”.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Any publications discussed above and throughout the text are provided solely for their disclosure prior to the filing date of the present application. Nothing herein is to be construed as an admission that the inventors are not entitled to antedate such disclosure by virtue of prior disclosure.

Gene regulation under drought stress is mediated by multiple transcriptional cascades (Zhu, 2002; Yamaguchi-Shinozaki and Shinozaki, 2006). In each of these cascades, a transcription factor gene is induced, which in turn activates or represses downstream target genes important for drought resistance. A number of stress regulated genes encode regulatory proteins, such as transcription factors, that are important in regulating the expression of still other downstream genes (Singh et al., 2002). In Arabidopsis, members of AP2/ERF, bZIP, NAC, HD-ZIP and MYB/MYC families as well as several classes of zinc finger domain proteins are induced by drought stress (Shinozaki et al., 2003; Zhang et al., 2004). In several cases, it has been shown that altering the expression of a transcription factor can alter stress resistance by activating downstream target genes. Examples of this are the CBFs/DREBs, NACs and RING-H2 zinc finger proteins (Jaglo-Ottosen et al., 1998; Kasuga et al., 1999; Hu et al., 2006; Ko et al., 2006).

Research has also focused on the identification of genetic factors that contribute to stress tolerance and on the genetic engineering of crop plants with increased stress tolerance. A number of genes have been identified whose expression or modified expression is associated with drought tolerance, via a variety of different mechanisms. For instance, transformed tobacco that express maize NADP-malic enzyme display increased water conservation and gained more mass per water consumed than wild-type plants (Laporte et al. 2002, J Exp Bot 53:699-705). Significant research effort has focused on the plant hormone abscisic acid (ABA), which is involved in adaptation to various environmental stresses. Transgenic tobacco and transgenic Arabidopsis that overexpress the enzyme 9-cis-epoxycarotenoid dioxygenase (NCED), which is key to ABA biosynthesis, display improved drought tolerance (Qin et al. 2002, Plant Physiol 128:544-51; Iuchi et al. 2001, Plant J 27:325-33). Drought tolerance is often linked to salt tolerance, since both are associated with regulation of osmotic potential and turgor. Accordingly, transgenic plants that overexpress a vacuolar H+ pump (H⁺-pyrophosphatase), which generates a proton gradient across the vacuolar membrane, display improved drought- and salt-stress, due to increased solute accumulation and water retention (Gaxiola et al. 2001, Proc Natl Acad Sci USA 98:11444-9). Trehalose also contributes to osmoprotection against environmental stress. Potato plants the mis-express trehalose-6-phosphate synthase, a key enzyme for trehalose biosynthesis, show increased drought tolerance (Yeo et al. 2000, Mol Cells 10:263-8).

Regulation of gene expression at the transcriptional level plays a crucial role in the development and physiological status of plants. With the discovery of small RNAs, increased attention has been focused on the importance of posttranscriptional gene regulation by small RNAs (Carrington and Ambros, 2003; Bartel, 2004; Tang, 2005). These small RNAs include 20-24 nucleotide (nt) microRNAs, 21 nt trans-acting siRNAs, ˜24 nt repeat-associated siRNAs and 21 or 24 nt nat-siRNAs. miRNAs are processed from hairpin precursors by the ribonuclease III-like enzyme Dicer. siRNAs differ from miRNAs in that they are generated from long double-stranded RNAs. Plant miRNAs are involved in various developmental processes, including flowering, leaf and root development, embryo development, and auxin signaling (Allen et al., 2005; Carrington and Ambros, 2003; Bartel, 2004; Jones-Rhoades et al., 2006). Recently, studies found that in Medicago truncatula, symbiotic nodule development is regulated by miR169 (Combier et al., 2006). microRNAs also play important roles in plant responses to biotic and abiotic stresses, such as sulfate and phosphate nutrient deprivation, and oxidative stress (Jones-Rhoades and Bartel, 2004; Fujii et al., 2005; Sunkar et al., 2006; Sunkar and Zhu, 2007). nat-siRNAs were demonstrated to regulate salt tolerance and disease resistance in Arabidopsis (Borsani et al. 2005; Katiyar-Agarwal et al., 2006). However, despite the importance of drought resistance, thus far no small RNAs have been reported to regulate drought stress responses.

Arabidopsis has served as a model system for the identification of genes that contribute to drought tolerance. For instance, researchers have identified numerous genes that are induced in response to water deprivation (e.g., Taji et al. 1999, Plant Cell Physiol 40:119-23; Ascenzi et al., 1997, Plant Mol Biol 34:629-41; Gosti et al. 1995, Mol Gen Genet. 246:10-18; Koizumi et al. 1993 Gene 129:175-82) and cis-acting DNA sequences called ABA responsive elements (ABREs) that control ABA or stress responsive gene expression (Giraudat et al. 1994, Plant Mol. Biol. 26: 1557).

Several drought tolerant mutants of Arabidopsis have been identified. These include the recessive mutants abh1 (Hugouvieux et al. 2001, Cell 106: 477), era1-2 (Pei et al. 1998, Science 282: 286) and abi1-1Ri (Gosti et al. 1999, Plant Cell 11:1897-1909). The mutants era1-2 and abh1 were identified by screening for seedlings hypersensitive to ABA, while the mutant abi1-1Ri was isolated as an intragenic suppressor of the ABA insensitive mutant abi1-1. Dominant drought tolerant mutants were identified by over-expressing ABF3, ABF4 (Kang et al. 2002, Plant Cell 14:343-357) or DREB1A (Kasuga, 1999 Nature Biotech 17: 287). ABF3 and ABF4 encode basic-region leucine zipper (bZIP) DNA binding proteins that bind specifically ABREs. DREB1A encodes a protein with an EREBP/AP2 DNA binding domain that binds to the dehydration-responsive element (DRE) essential for dehydration responsive gene expression (Liu et al. 1998, Plant Cell 10: 1391). A dominant drought tolerant phenotype in tobacco was obtained by over-expressing the soybean BiP gene (Alvim et al. 2001, Plant Physiol 126, 1042).

The disclosure demonstrates that NFYA5 is part of these drought stress-responsive transcriptional cascades. This transcriptional cascade is important for drought resistance because 35S::MIR169a and nfya5 mutant plants are hypersensitive to drought stress. The disclosure demonstrates that NFYA5 is post-transcriptionally downregulated by at least two miRNA molecules (mir169a and c). Furthermore, the disclosure demonstrates that expression or over-expression of an NFY5A polypeptide promotes drought tolerance. The expression or over-expression may be obtained through the use of (a) transcription regulation of a wild-type NFY5A (e.g., through the use of linking an nfy5a polynucoleotide to a constitutive or inducible promoter), (b) expression of a mutant nfy5a lacking a domain that interacts with mir169a or c; or (c) by inhibiting or knocking out the expression of either or both of mir169a and mir169c. Accordingly, the disclosure provides methods and compositions for generating transgenic plants having improved drought tolerance as well as methods for modifying drought tolerance in a plant.

Nuclear factor Y (NF-Y) is a universal transcription factor with high affinity and sequence specificity for the CCAAT box, a cis-element present in ˜25% of eukaryotic gene promoters. The NF-Y is a heterotrimeric complex composed of NF-YA (also known as CBF-B or HAP2), NF-YB (CBF-A or HAP3) and NF-YC (CBF-C or HAP5). In mammals, NF-YB and NF-YC tightly dimerize through a histone fold motif, then NF-YA associates prior to DNA binding, with the sequence-specific interaction of the trimer mediated by NF-YA (Mantovani, 1999). NF-YA and NF-YC subunits contain large domains rich in glutamines and hydrophobic residues that are important for activating transcription (Mantovani, 1999). In animals and yeast, each subunit of NF-Y is encoded by a single gene, whereas the Arabidopsis genome encodes 10 NF-YAs, 13 NF-YBs and 13 NF-YCs (Gusmaroli et al., 2002). It has been demonstrated that AtNFYB9 (LEC1) has a pivotal role in embryo development (Lee et al., 2003). Recently, overexpression of AtNFYB1 in Arabidopsis and maize was shown to significantly improve drought resistance and yield under drought stress conditions (Nelson et al., 2007). However, the biological roles of most of the NF-Y family members in plants are not understood.

This disclosure provides data showing that expression of AtNFYA5, a member of the Arabidopsis NF-YA family, is strongly induced by drought stress and ABA treatments. Promoter::GUS analysis shows that part of this induction occurred at the transcriptional level; however, transcriptional regulation alone could not explain the high level of NFYA5 transcript accumulation seen after stress or ABA treatment.

The disclosure further demonstrates that NFYA5 contains a target site for miR169, and miR169 expression was down-regulated by drought. When the expression of miR169 precursors were analyzed, two of the precursors, miR169a and miR169c, were downregulated by drought stress. Co-expression of miR169 and NFYA5 mRNAs suggested that miR169a was more efficient than miR169c at downregulating the NFYA5 mRNA. Thus, the results suggest that downregulation of miR169a by drought stress contributes to the high level induction of NFYA5 by drought and ABA. NFYA5 was highly expressed in vascular tissues and guard cells, and analysis of nfya5 knockout plants and miR169a or NFYA5 overexpression lines showed that NFYA5 was important in controlling stomatal aperture and drought resistance. Taken together, the results show that NFYA5 is important for drought resistance and it is regulated by drought stress at both transcriptional and posttranscriptional levels. Thus, The disclosure provides an isolated polynucleotide comprising: an NFY5A polynucleotide, homolog or otholog thereof operably linked to a heterologous promoter. In one embodiment, the NFY5A comprises at least 50%, 60%, 70%, 80%, 90%, 95%, 98%, 99%, 100% identity to a polynucleotide consisting of SEQ ID NO:1, 3, 5, or 7. In another embodiment, the NFY5A polynucleotide encodes a polypeptide comprising SEQ ID NO:2. In yet another embodiment, the heterologous promoter comprises a constitutive promoter or an inducible promoter. In one aspect, the constitutive promoter is a cauliflower mosaic virus 35S or 19S promoter or a plant ACT2 promoter or a plant ubiquitin promoter. In another aspect, the constitutive promoter is an Actin2 promoter derived from Arabidopsis thaliana. In yet another aspect, the inducible promoter comprises a light inducible promoter.

The disclosure also provides a plant cell transformed with a polynucleotide as set forth above.

The disclosure also provides a transgenic plant that expresses an NFY5A polynucleotide at a level higher than that of a wild-type plant and the transgenic plant has improved drought tolerance.

The disclosure further provides a transgenic plant that lacks the 3′UTR of a NFY5A gene, homolog or ortholog thereof.

The disclosure provides a transgenic plant that lacks a functional miR169 polynucleotide in the genome. For example, the transgenic plant can lack a functional miR169a or c polynucleotide in the genome.

The disclosure also provides a method of using the polynucleotide as described above to produce a plant which is resistant to drought, comprising the steps of introducing the polynucleotide or construct into a plant cell or into plant tissue, selecting for the presence of the polynucleotide molecule to produce a transgenic plant cell or transgenic plant tissue, and regenerating a plant from the transgenic plant cell or transgenic plant tissue, whereby a drought resistant plant is generated. In one embodiment, the polynucleotide comprises a sequence encoding an NFY5A polypeptide operably linked to a heterolgous promoter. In another embodiment, the polynucleotide comprises a construct that reduces the expression of an mir169a or c or which provides a complement of an mir169a or c or which knocks out mir169a or c in a plant cell.

The disclosure also provides a method for identifying agents useful for modifying drought resistance in a plant comprising: contacting a plant that contains an NFY5A gene with an agent; measuring a change in expression of the gene or the amount of mRNA transcript present in the cell; wherein a change in expression or transcript levels compared to a control is indicative of an agent that modifies drought resistance.

In general, the methods of the disclosure involve incorporating the desired form of the NFYA5 polynucleotide into a plant expression vector for transformation of plant cells, and the NFYA5 polypeptide is expressed or overexpressed in the host plant either constitutively or inducibly. In another embodiment, the disclosure provides for NFYA5 polypeptides the are encoded by a polynucleotide that does not bind a siRNA or miRNA molecule (e.g., miR169 molecule). Because a wild-type NFYA5 is inhibited by mrR169, an inhibition-resistant NFYA5 polynucleotide would provide a method of modulating drought tolerance (i.e., improve drought tolerance) compared to a wild-type. Furthermore, a knockout transgenic plant lacking a sequence encoding or providing an miRNA (e.g., miR169) would also provide a drough tolerant plant.

NFYA5 nucleic acids and polypeptides may be used in the generation of genetically modified plants having a modified (e.g. an improved) drought tolerance phenotype. Such plants may further display increased tolerance to other abiotic stresses, particular salt-stress and freezing, as responses to these stresses and drought stress are mediated by ABA (Thomashow, 1999 Annu. Revl Plant Physiol. Plant Mol. Biol. 50: 571; Cushman and Bohnert, 2000, Curr. Opin. Plant Biol. 3: 117; Kang et al. 2002, Plant Cell 14:343-357; Quesada et al. 2000, Genetics 154: 421; Kasuga et al. 1999, Nature Biotech. 17: 287-291).

The methods described herein are generally applicable to all plants. Drought tolerance is an important trait in almost any agricultural crop; most major agricultural crops, including corn, wheat, soybeans, cotton, alfalfa, sugar beets, onions, tomatoes, and beans, are susceptible to drought stress. Although the specific examples provided below were performed in a select species, the NFYA5 gene (or an ortholog, variant or fragment thereof) may be expressed in any type of plant. The disclosure can be used to confer drought tolerance in fruit- and vegetable-bearing plants, plants used in the cut flower industry, grain-producing plants, oil-producing plants, nut-producing plants, crops including corn (Zea mays), soybean (Glycine max) cotton (Gossypium), tomato (Lycopersicum esculentum), alfalfa (Medicago sativa), flax (Linum usitatissimum), tobacco (Nicotiana), and turfgrass (Poaceae family), and other forage crops, among others.

The skilled artisan will recognize that a wide variety of transformation techniques exist in the art, and new techniques are continually becoming available. Any technique that is suitable for the target host plant can be employed within the scope of the disclosure. For example, the constructs can be introduced in a variety of forms including, but not limited to as a strand of DNA, in a plasmid, or in an artificial chromosome. The introduction of the constructs into the target plant cells can be accomplished by a variety of techniques, including, but not limited to Agrobacterium-mediated transformation, electroporation, microinjection, microprojectile bombardment calcium-phosphate-DNA co-precipitation or liposome-mediated transformation of a heterologous nucleic acid. The transformation of the plant is preferably permanent, i.e. by integration of the introduced expression constructs into the host plant genome, so that the introduced constructs are passed onto successive plant generations. Depending upon the intended use, a heterologous nucleic acid construct comprising an NFYA5 polynucleotide may encode the entire protein or a biologically active portion thereof.

In one embodiment, binary Ti-based vector systems may be used to transfer polynucleotides. Standard Agrobacterium binary vectors are known to those of skill in the art, and many are commercially available (e.g., pBI121 Clontech Laboratories, Palo Alto, Calif.).

As used herein, the term “vector” refers to a nucleic acid construct designed for transfer between different host cells. An “expression vector” refers to a vector that has the ability to incorporate and express heterologous DNA fragments in a foreign cell. Many prokaryotic and eukaryotic expression vectors are commercially available. Selection of appropriate expression vectors is within the knowledge of those having skill in the art.

A “heterologous” nucleic acid construct or sequence has a portion of the sequence that is not native to the plant cell in which it is expressed. Heterologous, with respect to a control sequence refers to a control sequence (i.e. promoter or enhancer) that does not function in nature to regulate the same gene the expression of which it is currently regulating. Generally, heterologous nucleic acid sequences are not endogenous to the cell or part of the genome in which they are present, and have been added to the cell, by infection, transfection, microinjection, electroporation, or the like. A “heterologous” nucleic acid construct may contain a control sequence/DNA coding sequence combination that is the same as, or different from a control sequence/DNA coding sequence combination found in the native plant.

As used herein, the term “gene” means the segment of DNA involved in producing a polypeptide chain, which may or may not include regions preceding and following the coding region, e.g., at 5′ untranslated (5′ UTR) or “leader” sequences and/or 3′UTR or “trailer” sequences, as well as intervening sequences (introns) between individual coding segments (exons) and non-transcribed regulatory sequence.

As used herein, “recombinant” includes reference to a cell or vector, that has been modified by the introduction of a heterologous nucleic acid sequence or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention.

As used herein, the term “gene expression” refers to the process by which a polypeptide is produced based on the nucleic acid sequence of a gene. The process includes both transcription and translation; accordingly, “expression” may refer to either a polynucleotide or polypeptide sequence, or both. Sometimes, expression of a polynucleotide sequence will not lead to protein translation. “Over-expression” refers to increased expression of a polynucleotide and/or polypeptide sequence relative to its expression in a wild-type or other plant and may relate to a naturally-occurring or non-naturally occurring sequence. “Ectopic expression” refers to expression at a time, place, and/or increased level that does not naturally occur in the non-altered or wild-type plant. “Under-expression” refers to decreased expression of a polynucleotide and/or polypeptide sequence, generally of an endogenous gene, relative to its expression in a wild-type plant. The terms “mis-expression” and “altered expression” encompass over-expression, under-expression, and ectopic expression.

The term “introduced” in the context of inserting a polynucleotide into a cell, means “transfection”, or “transformation” or “transduction” and includes reference to the incorporation of a polynucleotide into a eukaryotic or prokaryotic cell where the polynucleotide may be incorporated into the genome of the cell (for example, chromosome, plasmid, plastid, or mitochondrial DNA), converted into an autonomous replicon, or transiently expressed (for example, transfected mRNA).

As used herein, a “plant cell” refers to any cell derived from a plant, including cells from undifferentiated tissue (e.g., callus) as well as plant seeds, pollen, progagules and embryos.

The disclosure encompasses modified plants and plant compositions including whole plants, shoot vegetative organs/structures (e.g., leaves, stems and tubers), roots, flowers and floral organs/structures (e.g., bracts, sepals, petals, stamens, carpels, anthers and ovules), seed (including embryo, endosperm, and seed coat) and fruit (the mature ovary), plant tissue (e.g., vascular tissue, ground tissue, and the like) and cells (e.g., guard cells, egg cells, and the like), and progeny of same. The class of plants that can be used in the method of the disclosure is generally as broad as the class of higher and lower plants amenable to transformation techniques, including angiosperms (monocotyledonous and dicotyledonous plants), gymnosperms, ferns, horsetails, psilophytes, lycophytes, bryophytes, and multicellular algae. “Plant tissue” includes differentiated and undifferentiated tissues or plants, including but not limited to roots, stems, shoots, leaves, pollen, seeds, tumor tissue and various forms of cells and culture such as single cells, protoplast, embryos, and callus tissue. The plant tissue may be in plants or in organ, tissue or cell culture.

The disclosure is not limited to any plant species. Plants species contemplated include, but are not limited to, alfalfa, aster, barley, begonia, beet, canola, cantaloupe, carrot, chrysanthemum, clover, corn, cotton, cucumber, delphinium, grape, lawn and turf grasses, lettuce, pea, peppermint, rice, rutabaga, sorghum, sugar beet, sunflower, tobacco, tomatillo, tomato, turnip, wheat, and zinnia.

As used herein, the terms “native” and “wild-type” relative to a given plant trait or phenotype refers to the form in which that trait or phenotype is found in the same variety of plant in nature.

As used herein, the term “modified” regarding a plant trait, refers to a change in the phenotype of a transgenic plant relative to the similar non-transgenic plant. An “interesting phenotype (trait)” with reference to a transgenic plant refers to an observable or measurable phenotype demonstrated by a T1 and/or subsequent generation plant, which is not displayed by the corresponding non-transgenic (i.e., a genotypically similar plant that has been raised or assayed under similar conditions). An interesting phenotype may represent an improvement in the plant or may provide a means to produce improvements in other plants. An “improvement” is a feature that may enhance the utility of a plant species or variety by providing the plant with a unique and/or novel quality.

An “altered drought tolerance phenotype” refers to detectable change in the ability of a genetically modified plant to withstand low-water conditions compared to the similar, but non-modified plant. In general, improved (increased) drought tolerance phenotypes (i.e., ability to a plant to survive in low-water conditions that would normally be deleterious to a plant) are of interest.

As used herein, a “mutant” polynucleotide or gene differs from the corresponding wild type polynucleotide or gene either in terms of sequence or expression, where the difference contributes to a modified plant phenotype or trait. Relative to a plant or plaint line, the term “mutant” refers to a plant or plant line which has a modified plant phenotype or trait, where the modified phenotype or trait is associated with the modified expression of a wild type polynucleotide or gene.

As used herein, the term “T1” refers to the generation of plants from the seed of T0 plants. The T1 generation is the first set of transformed plants that can be selected by application of a selection agent, e.g., an antibiotic or herbicide, for which the transgenic plant contains the corresponding resistance gene. The term “T2” refers to the generation of plants by self-fertilization of the flowers of T1 plants, previously selected as being transgenic.

As used herein, the term “plant part” includes any plant organ or tissue, including, without limitation, seeds, embryos, meristematic regions, callus tissue, leaves, roots, shoots, gametophytes, sporophytes, pollen, and microspores. Plant cells can be obtained from any plant organ or tissue and cultures prepared therefrom. The class of plants which can be used in the methods of the present invention is generally as broad as the class of higher plants amenable to transformation techniques, including both monocotyledenous and dicotyledenous plants.

As used herein, “transgenic plant” includes reference to a plant that comprises within its genome a heterologous polynucleotide. The heterologous polynucleotide can be either stably integrated into the genome, or can be extra-chromosomal. Preferably, the polynucleotide of the disclosure is stably integrated into the genome such that the polynucleotide is passed on to successive generations. A plant cell, tissue, organ, or plant into which the heterologous polynucleotides have been introduced is considered “transformed”, “transfected”, or “transgenic”. Direct and indirect progeny of transformed plants or plant cells that also contain the heterologous polynucleotide are also considered transgenic.

A “protein” or “polypeptide”, which terms are used interchangeably herein, comprises one or more chains of chemical building blocks called amino acids that are linked together by chemical bonds called peptide bonds. A “native” or “wild-type” protein, enzyme, polynucleotide, gene, or cell, means a protein, enzyme, polynucleotide, gene, or cell that occurs in nature.

An “amino acid sequence” is a polymer of amino acids (a protein, polypeptide, etc.) or a character string representing an amino acid polymer, depending on context. The amino acids which occur in the various amino acid sequences referred to in the specification have their usual three- and one-letter abbreviations routinely used in the art: A, Ala, Alanine; C, Cys, Cysteine; D, Asp, Aspartic Acid; E, Glu, Glutamic Acid; F, Phe, Phenylalanine; G, Gly, Glycine; H, His, Histidine; I, Ile, Isoleucine; K, Lys, Lysine; L, Leu, Leucine; M, Met, Methionine; N, Asn, Asparagine; P, Pro, Proline; Q, Gln, Glutamine; R, Arg, Arginine; S, Ser, Serine; T, Thr, Threonine; V, Val, Valine; W, Try, Tryptophan; Y, Tyr, Tyrosine.

“Conservative amino acid substitution” or, simply, “conservative variations” of a particular sequence refers to the replacement of one amino acid, or series of amino acids, with essentially identical amino acid sequences. One of skill will recognize that individual substitutions, deletions or additions which alter, add or delete a single amino acid or a percentage of amino acids in an encoded sequence result in “conservative variations” where the alterations result in the deletion of an amino acid, addition of an amino acid, or substitution of an amino acid with a chemically similar amino acid.

Conservative substitution tables providing functionally similar amino acids are well known in the art. For example, one conservative substitution group includes Alanine (A), Serine (S), and Threonine (T). Another conservative substitution group includes Aspartic acid (D) and Glutamic acid (E). Another conservative substitution group includes Asparagine (N) and Glutamine (Q). Yet another conservative substitution group includes Arginine (R) and Lysine (K). Another conservative substitution group includes Isoleucine, (I) Leucine (L), Methionine (M), and Valine (V). Another conservative substitution group includes Phenylalanine (F), Tyrosine (Y), and Tryptophan (W).

Thus, “conservative amino acid substitutions” of a listed polypeptide of the disclosure include substitutions of a percentage, typically less than 10%, of the amino acids of the polypeptide, with a conservatively selected amino acid of the same conservative substitution group. Accordingly, a conservatively substituted variation of a polypeptide of the disclosure can contain 100, 75, 50, 25, or 10 substitutions with a conservatively substituted variation of the same conservative substitution group.

“Conservative variants” are proteins or enzymes in which a given amino acid residue has been changed without altering overall conformation and function of the protein or enzyme, including, but not limited to, replacement of an amino acid with one having similar properties, including polar or non-polar character, size, shape and charge. Amino acids other than those indicated as conserved may differ in a protein or enzyme so that the percent protein or amino acid sequence similarity between any two proteins of similar function may vary and can be, for example, at least 30%, at least 50%, at least 70%, at least 80%, or at least 90%, as determined according to an alignment scheme. As referred to herein, “sequence similarity” means the extent to which nucleotide or protein sequences are related. The extent of similarity between two sequences can be based on percent sequence identity and/or conservation. “Sequence identity” herein means the extent to which two nucleotide or amino acid sequences are invariant. “Sequence alignment” means the process of lining up two or more sequences to achieve maximal levels of identity (and, in the case of amino acid sequences, conservation) for the purpose of assessing the degree of similarity. Numerous methods for aligning sequences and assessing similarity/identity are known in the art such as, for example, the Cluster Method, wherein similarity is based on the MEGALIGN algorithm, as well as BLASTN, BLASTP, and FASTA (Lipman and Pearson, 1985; Pearson and Lipman, 1988). When using all of these programs, the preferred settings are those that results in the highest sequence similarity.

As used herein, “percent identity” with respect to a specified subject sequence, or a specified portion thereof, is defined as the percentage of nucleotides or amino acids in the candidate derivative sequence identical with the nucleotides or amino acids in the subject sequence (or specified portion thereof), after aligning the sequences and introducing gaps, if necessary to achieve the maximum percent sequence identity, as generated by the program WU-BLAST-2.0a19 (Altschul et al., J. Mol. Biol. (1990) 215:403-410; website at blast.wust1.edu/blast/README.html) with search parameters set to default values. The HSP S and HSP S2 parameters are dynamic values and are established by the program itself depending upon the composition of the particular sequence and composition of the particular database against which the sequence of interest is being searched. A “% identity value” is determined by the number of matching identical nucleotides or amino acids divided by the sequence length for which the percent identity is being reported. “Percent amino acid similarity” is determined by doing the same calculation as for determining percent amino acid sequence identity, but including conservative amino acid substitutions in addition to identical amino acids in the computation.

Non-conservative modifications of a particular polypeptide are those which substitute any amino acid not characterized as a conservative substitution. For example, any substitution which crosses the bounds of the six groups set forth above. These include substitutions of basic or acidic amino acids for neutral amino acids, (e.g., Asp, Glu, Asn, or Gln for Val, Ile, Leu or Met), aromatic amino acid for basic or acidic amino acids (e.g., Phe, Tyr or Trp for Asp, Asn, Glu or Gln) or any other substitution not replacing an amino acid with a like amino acid. Basic side chains include lysine (K), arginine (R), histidine (H); acidic side chains include aspartic acid (D), glutamic acid (E); uncharged polar side chains include glycine (G), asparagine (N), glutamine (Q), serine (S), threonine (T), tyrosine (Y), cysteine (C); nonpolar side chains include alanine (A), valine (V), leucine (L), isoleucine (I), proline (P), phenylalanine (F), methionine (M), tryptophan (W); beta-branched side chains include threonine (T), valine (V), isoleucine (I); aromatic side chains include tyrosine (Y), phenylalanine (F), tryptophan (W), histidine (H).

It is understood that the addition of sequences which do not alter the activity of a polypeptide encoded by a particular polynucleotide, such as the addition of a non-functional or non-coding sequence, is a conservative variation of the basic polynucleotide.

One of skill in the art will appreciate that many conservative variations of the nucleic acid constructs which are disclosed yield a functionally identical construct. For example, as discussed above, owing to the degeneracy of the genetic code, “silent substitutions” (i.e., substitutions in a nucleic acid sequence which do not result in an alteration in an encoded polypeptide) are an implied feature of every nucleic acid sequence which encodes an amino acid. Similarly, “conservative amino acid substitutions,” in one or a few amino acids in an amino acid sequence are substituted with different amino acids with highly similar properties, are also readily identified as being highly similar to a disclosed construct. Such conservative variations of each disclosed sequence are a feature of the polypeptides provided herein.

A polynucleotide, polypeptide, or other component is “isolated” or “purified” when it is partially or completely separated from components with which it is normally associated (other proteins, nucleic acids, cells, synthetic reagents, etc.). A polynucleotide or polypeptide is “recombinant” when it is artificial or engineered, or derived from an artificial or engineered protein or nucleic acid. For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide that does not appear in nature, for example a variant of a naturally occurring gene, is recombinant.

Any vector including a plasmid, cosmid, phage or Agrobacterium binary vector in double or single stranded linear or circular form which may or may not be self transmissible or mobilizable, and which can transform prokaryotic or eukaryotic host either by integration into the cellular genome or exist extrachromosomally (e.g. autonomous replicating plasmid with an origin of replication) can be used in the methods and compositions of the disclosure.

Specifically included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells).

A polynucleotide in a vector is under the control of, and operably linked to, an appropriate promoter or other regulatory elements for transcription in a host cell such as a microbial, e.g. bacterial, or plant cell. The vector may be a bi-functional expression vector which functions in multiple hosts. In the case of genomic DNA, this may contain its own promoter or other regulatory elements and in the case of cDNA this may be under the control of an appropriate promoter or other regulatory elements for expression in the host cell.

As used herein, “constitutive promoter” refers to a promoter that facilitates the expression of an operably linked coding polynucleotide or polynucleotide of interest at a substantially continuous or basal level. An inducible promoter is a promoter that can be induced temporally or by contact such that the level of expression is changed over a specific period of time. Examples of promoters suitable for use with a NFY5A polynucleotide or fragment of the disclosure include regulatory sequences from fatty acid desaturase genes, alcohol dehydrogenase promoter from corn, light inducible promoters such as the ribulose bisphosphate carboxylase small subunit gene, major chlorophyll a/b binding protein gene promoters, the 19S promoter of cauliflower mosaic virus (CaMV, the −46 by Cauliflower Mosaic Virus (CaMV) 35S minimal promoter which is expressed at low (basal) levels in most plant tissues. Tissue-specific promoters, such root-specific promoters or root cortex specific promoters, are also contemplated. Non-limiting examples of seed-specific promoters include napin, phaseolin, oleosin, and cruciferin promoters. Other suitable plant promoters include those known to persons of ordinary skill in the art.

The disclosure includes the terms “regulatory sequence,” “control element,” and “expression control sequence” to refer to polynucleotide domains comprising sequences that influence transcription initiation and rate. Regulatory sequences include, but are not limited to, promoters, promoter control elements, protein binding domains, 5′ and 3′ untranslated regions (UTRs), transcriptional start sites, termination sequences, polyadenylation sequences, introns, and other regulatory sequences that reside within a coding sequence.

The terms “operably linked” and “operably associated” are used interchangeably herein to broadly refer to a chemical or physical coupling (directly or indirectly) of two otherwise distinct domains in a molecule, wherein each domain has independent biological function. For example, operably linked refers to the functional connection between a regulatory sequence and the polynucleotide regulated by the regulatory sequence. For example, an operably linked 5NFYA5 polynucleotide or fragment of the disclosure can comprise a 5NFYA5 polynucleotide or fragment operably linked to a promoter, which is in turn operably linked to a polynucleotide encoding a polypeptide or inhibitory nucleic acid molecule to be expressed.

A promoter is an expression control sequence composed of a region of a DNA molecule, typically within 100 nucleotides upstream of the point at which transcription starts (generally near the initiation site for RNA polymerase II). Promoters are involved in recognition and binding of RNA polymerase and other proteins to initiate and modulate transcription. To bring a coding sequence under the control of a promoter, it typically is necessary to position the translation initiation site of the translational reading frame of the polypeptide between one and about fifty nucleotides downstream of the promoter.

A minimal promoter comprises only a necessary amount of sequence for assembly of a transcription complex required for transcription initiation. Minimal promoters typically include a “TATA box” element that may be located between about 15 and about 35 nucleotides upstream from the site of transcription initiation. Minimal promoters may also include a “CCAAT box” element, which can be located between about 40 and about 200 nucleotides, typically about 60 to about 120 nucleotides, upstream from the transcription start site.

Arabidopsis NFYA5 nucleic acid (cDNA) sequence is provided in SEQ ID NO:1, 3, 5, or 7. The corresponding protein sequence is provided in SEQ ID NO:2, 4, 6, and 8, respectively.

As used herein, the term “NFYA5 polypeptide” refers to a full-length NFYA5 protein or a fragment, derivative (variant), or ortholog thereof that is “functionally active,” meaning that the protein fragment, derivative, or ortholog exhibits one or more or the functional activities associated with the polypeptide of SEQ ID NO: 2, 4, 6, or 8. In one embodiment, a functionally active NFYA5 polypeptide causes an altered drought tolerance phenotype when over-expressed or mis-expressed in a plant. In a further embodiment, mis- or over-expression of the functionally active NFYA5 polypeptide causes improved drought tolerance. In another embodiment, a functionally active NFYA5 polypeptide is capable of rescuing defective (including deficient) endogenous NFYA5 activity when expressed in a plant or in plant cells; the rescuing polypeptide may be from the same or from a different species as that with defective activity. In another embodiment, a functionally active fragment of a full length NFYA5 polypeptide (i.e., a native polypeptide having the sequence of SEQ ID NO: 2, 4, 6, or 8 or a naturally occurring ortholog thereof) retains one of more of the biological properties associated with the full-length NFYA5 polypeptide, such as signaling activity, binding activity, catalytic activity, or cellular or extra-cellular localizing activity. A NFYA5 fragment preferably comprises a NFYA5 domain, such as a C- or N-terminal or catalytic domain, among others, and preferably comprises at least 10, preferably at least 20, more preferably at least 25, and most preferably at least 50 contiguous amino acids of a NFYA5 protein. Functional domains can be identified using the PFAM program (Bateman A et al., Nucleic Acids Res (1999) 27:260-262; website at pfam.wust1.edu). Functionally active variants of full-length NFYA5 polypeptides or fragments thereof include polypeptides with amino acid insertions, deletions, or substitutions that retain one of more of the biological properties associated with the full-length NFYA5 polypeptide. In some cases, variants are generated that change the post-translational processing of a NFYA5 polypeptide. For instance, variants may have altered protein transport or protein localization characteristics or altered protein half-life compared to the native polypeptide.

As used herein, the term “NFYA5 polynucleotide” encompasses polynucleotides with a sequence as set forth in or complementary to the sequence provided in SEQ ID NO: 1, as well as functionally active fragments, derivatives, or orthologs thereof A NFYA5 polynucleotide of this disclosure may be DNA, derived from genomic DNA or cDNA, or RNA or a combination thereof.

In one embodiment, a NFYA5 polynucleotide encodes or is complementary to a nucleic acid that encodes a functionally active NFYA5 polypeptide (e.g., a polypeptide comprising SEQ ID NO: 2, 4, 6, or 8 or a functional fragment thereof). Included within this definition is genomic DNA that serves as a template for a primary RNA transcript (i.e., an mRNA precursor) that requires processing, such as splicing, before encoding the functionally active NFYA5 polypeptide. A NFYA5 polynucleotide can include other non-coding sequences, which may or may not be transcribed; such sequences include 5′ and 3′ UTRs, polyadenylation signals and regulatory sequences that control gene expression, among others, as are known in the art. Some polypeptides require processing events, such as proteolytic cleavage, covalent modification, etc., in order to become fully active. Accordingly, functionally active nucleic acids may encode the mature or the pre-processed NFYA5 polypeptide, or an intermediate form. A NFYA5 polynucleotide can also include heterologous coding sequences, for example, sequences that encode a marker included to facilitate the purification of the fused polypeptide, or a transformation marker.

In another embodiment, a functionally active NFYA5 polynucleotide is capable of being used in the generation of loss-of-function NFYA5 phenotypes, for instance, via antisense suppression, co-suppression, and the like.

In one embodiment, a NFYA5 polynucleotide used in the methods of this disclosure comprises a nucleic acid sequence that encodes or is complementary to a sequence that encodes a NFYA5 polypeptide having at least 50%, 60%, 70%, 75%, 80%, 85%, 90%, 95% or more sequence identity to the polypeptide sequence presented in SEQ ID NO: 2, 4, 6, or 8.

In another embodiment a NFYA5 polypeptide of the disclosure comprises a polypeptide sequence with at least 50% or 60% identity to the NFYA5 polypeptide sequence of SEQ ID NO: 2, 4, 6, or 8, and may have at least 70%, 80%, 85%, 90% or 95% or more sequence identity to the NFYA5 polypeptide sequence of SEQ ID NO: 2, 4, 6, or 8. In another embodiment, a NFYA5 polypeptide comprises a polypeptide sequence with at least 50%, 60%, 70%, 80%, 85%, 90% or 95% or more sequence identity to a functionally active fragment of the polypeptide presented in SEQ ID NO: 2, 4, 6, or 8.

In another aspect, a NFYA5 polynucleotide comprises a sequence that is at least 50% to 60% identical over its entire length to the NFYA5 polynucleotide sequence presented as SEQ ID NO:1, 3, 5, or 7, or nucleic acid sequences that are complementary to such a NFYA5 sequence, and may comprise at least 70%, 80%, 85%, 90% or 95% or more sequence identity to the NFYA5 sequence presented as SEQ ID NO: 1 or a functionally active fragment thereof, or complementary sequences.

The disclosure also encompasses nucleic acid molecules capable of hybridizing to a polynucleotide consisting of sequence of SEQ ID NO:1, 3, 5, or 7 and encoding a polypeptide that provides drought tolerance to a plant. The stringency of hybridization can be controlled by temperature, ionic strength, pH, and the presence of denaturing agents such as formamide during hybridization and washing. Conditions routinely used are well known (see, e.g., Current Protocol in Molecular Biology, Vol. 1, Chap. 2.10, John Wiley & Sons, Publishers (1994); Sambrook et al., supra). In some embodiments, a nucleic acid molecule of the disclosure is capable of hybridizing to a nucleic acid molecule containing the nucleotide sequence of SEQ ID NO: 1 under stringent hybridization conditions that comprise: prehybridization of filters containing nucleic acid for 8 hours to overnight at 65° C. in a solution comprising 6× single strength citrate (SSC) (1×SSC is 0.15 M NaCl, 0.015 M Na citrate; pH 7.0), 5×Denhardt's solution, 0.05% sodium pyrophosphate and 100 ng/ml herring sperm DNA; hybridization for 18-20 hours at 65° C. in a solution containing 6×SSC, 1×Denhardt's solution, 100 ng/ml yeast tRNA and 0.05% sodium pyrophosphate; and washing of filters at 65° C. for 1 h in a solution containing 0.2×SSC and 0.1% SDS (sodium dodecyl sulfate). In other embodiments, moderately stringent hybridization conditions are used that comprise: pretreatment of filters containing nucleic acid for 6 h at 40° C. in a solution containing 35% formamide, 5×SSC, 50 mM Tris-HC1 (pH 7.5), 5 mM EDTA, 0.1% PVP, 0.1% Ficoll, 1% BSA, and 500 ng/ml denatured salmon sperm DNA; hybridization for 18-20 h at 40° C. in a solution containing 35% formamide, 5×SSC, 50 mM Tris-HCl (pH 7.5), 5 mM EDTA, 0.02% PVP, 0.02% Ficoll, 0.2% BSA, 100 ng/ml salmon sperm DNA, and 10% (wt/vol) dextran sulfate; followed by washing twice for 1 hour at 55° C. in a solution containing 2×SSC and 0.1% SDS. Alternatively, low stringency conditions can be used that comprise: incubation for 8 hours to overnight at 37° C. in a solution comprising 20% formamide, 5×SSC, 50 mM sodium phosphate (pH 7.6), 5×Denhardt's solution, 10% dextran sulfate, and 20 ng/ml denatured sheared salmon sperm DNA; hybridization in the same buffer for 18 to 20 hours; and washing of filters in 1×SSC at about 37° C. for 1 hour.

The methods of the disclosure may use orthologs of the Arabidopsis NFYA5. Methods of identifying the orthologs in other plant species are known in the art. Normally, orthologs in different species retain the same function, due to presence of one or more protein motifs and/or 3-dimensional structures. In evolution, when a gene duplication event follows speciation, a single gene in one species, such as Arabidopsis, may correspond to multiple genes (paralogs) in another. As used herein, the term “orthologs” encompasses paralogs. When sequence data is available for a particular plant species, orthologs are generally identified by sequence homology analysis, such as BLAST analysis, usually using protein bait sequences. Sequences are assigned as a potential ortholog if the best hit sequence from the forward BLAST result retrieves the original query sequence in the reverse BLAST (Huynen M A and Bork P, Proc Natl Acad Sci (1998) 95:5849-5856; Huynen M A et al., Genome Research (2000) 10:1204-1210). Programs for multiple sequence alignment, such as CLUSTAL (Thompson J D et al, Nucleic Acids Res (1994) 22:4673-4680) may be used to highlight conserved regions and/or residues of orthologous proteins and to generate phylogenetic trees. In a phylogenetic tree representing multiple homologous sequences from diverse species (e.g., retrieved through BLAST analysis), orthologous sequences from two species generally appear closest on the tree with respect to all other sequences from these two species. Structural threading or other analysis of protein folding (e.g., using software by ProCeryon, Biosciences, Salzburg, Austria) may also identify potential orthologs. Nucleic acid hybridization methods may also be used to find orthologous genes and are when sequence data are not available. Degenerate PCR and screening of cDNA or genomic DNA libraries are common methods for finding related gene sequences and are well known in the art (see, e.g., Sambrook, supra; Dieffenbach C and Dveksler G (Eds.) PCR Primer: A Laboratory Manual, Cold Spring Harbor Laboratory Press, NY, 1989). For instance, methods for generating a cDNA library from the plant species of interest and probing the library with partially homologous gene probes are described in Sambrook et al. A highly conserved portion of the Arabidopsis NFYA5 coding sequence may be used as a probe. NFYA5 ortholog nucleic acids may hybridize to the nucleic acid of SEQ ID NO: 1 under high, moderate, or low stringency conditions. After amplification or isolation of a segment of a putative ortholog, that segment may be cloned and sequenced by standard techniques and utilized as a probe to isolate a complete cDNA or genomic clone. Alternatively, it is possible to initiate an EST project to generate a database of sequence information for the plant species of interest. In another approach, antibodies that specifically bind known NFYA5 polypeptides are used for ortholog isolation. Western blot analysis can determine that a NFYA5 ortholog (i.e., an orthologous protein) is present in a crude extract of a particular plant species. When reactivity is observed, the sequence encoding the candidate ortholog may be isolated by screening expression libraries representing the particular plant species. Expression libraries can be constructed in a variety of commercially available vectors, including lambda gt11, as described in Sambrook, et al., supra. Once the candidate ortholog(s) are identified by any of these means, candidate orthologous sequence are used as bait (the “query”) for the reverse BLAST against sequences from Arabidopsis or other species in which NFYA5 nucleic acid and/or polypeptide sequences have been identified.

NFYA5 polynucleotides and polypeptides may be obtained using any available method. For instance, techniques for isolating cDNA or genomic DNA sequences of interest by screening DNA libraries or by using polymerase chain reaction (PCR), as previously described, are well known in the art. Alternatively, polynucleotides and oligonucleotides may be synthesized. Any known method, such as site directed mutagenesis (Kunkel T A et al., Methods Enzymol. (1991) 204:125-39), may be used to introduce desired changes into a cloned nucleic acid.

As will be understood by those of skill in the art, it can be advantageous to modify a coding sequence to enhance its expression in a particular host. The genetic code is redundant with 64 possible codons, but most organisms preferentially use a subset of these codons. The codons that are utilized most often in a species are called optimal codons, and those not utilized very often are classified as rare or low-usage codons (see, e.g., Zhang et al. (1991) Gene 105:61-72). Codons can be substituted to reflect the preferred codon usage of the host, a process sometimes called “codon optimization” or “controlling for species codon bias.”

Optimized coding sequences containing codons preferred by a particular prokaryotic or eukaryotic host (see also, Murray et al. (1989) Nucl. Acids Res. 17:477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, preferred stop codons for S. cerevisiae and mammals are UAA and UGA, respectively. The preferred stop codon for monocotyledonous plants is UGA, whereas insects and E. coli prefer to use UAA as the stop codon (Dalphin et al. (1996) Nucl. Acids Res. 24: 216-218). Methodology for optimizing a nucleotide sequence for expression in a plant is provided, for example, in U.S. Pat. No. 6,015,891, and the references cited therein.

The optimal procedure for transformation of plants with Agrobacterium vectors will vary with the type of plant being transformed. Exemplary methods for Agrobacterium-mediated transformation include transformation of explants of hypocotyl, shoot tip, stem or leaf tissue, derived from sterile seedlings and/or plantlets. Such transformed plants may be reproduced sexually, or by cell or tissue culture. Agrobacterium transformation has been previously described for a large number of different types of plants and methods for such transformation may be found in the scientific literature.

Expression (including transcription and translation) of NFYA5 may be regulated with respect to the level of expression, the tissue type(s) where expression takes place and/or developmental stage of expression. A number of heterologous regulatory sequences (e.g., promoters and enhancers) are available for controlling the expression of a NFYA5 nucleic acid. These include constitutive, inducible and regulatable promoters, as well as promoters and enhancers that control expression in a tissue- or temporal-specific manner. Exemplary constitutive promoters include the raspberry E4 promoter (U.S. Pat. Nos. 5,783,393 and 5,783,394), the 35S CaMV (Jones J D et al, (1992) Transgenic Res 1:285-297), the CsVMV promoter (Verdaguer B et al., Plant Mol Biol (1998) 37:1055-1067) and the melon actin promoter (published PCT application WO0056863). Exemplary tissue-specific promoters include the tomato E4 and E8 promoters (U.S. Pat. No. 5,859,330) and the tomato 2AII gene promoter (Van Haaren M J J et al., Plant Mol Bio (1993) 21:625-640).

In one embodiment, NFYA5 expression is under control of regulatory sequences from genes whose expression is associated with drought stress. Promoters from drought stress inducible genes in other species could be used also. Examples are the rab17, ZmFer1 and ZmFer2 genes from maize (Bush et al, 1997 Plant J 11:1285; Fobis-Loisy, 1995 Eur J Biochem 231:609), the tdi-65 gene from tomato (Harrak, 2001 Genome 44:368), the His1 gene of tobacco (Wei and O'Connell, 1996 Plant Mol Biol 30:255), the Vupat1 gene from cowpea (Matos, 2001 FEBS Lett 491:188), and CDSP34 from Solanum tuberosum (Gillet et al, 1998 Plant J 16:257).

In yet another aspect, in some cases it may be desirable to inhibit the expression of endogenous NFYA5 in a host cell. Exemplary methods for practicing this aspect of the disclosure include, but are not limited to antisense suppression (Smith, et al., Nature (1988) 334:724-726; van der Krol et al., Biotechniques (1988) 6:958-976); co-suppression (Napoli, et al, Plant Cell (1990) 2:279-289); ribozymes (PCT Publication WO 97/1032S); and combinations of sense and antisense (Waterhouse, et al., Proc. Natl. Acad. Sci. USA (1998) 95:13959-13964). Methods for the suppression of endogenous sequences in a host cell typically employ the transcription or transcription and translation of at least a portion of the sequence to be suppressed. Such sequences may be homologous to coding as well as non-coding regions of the endogenous sequence. Antisense inhibition may use the entire cDNA sequence (Sheehy et al., Proc. Natl. Acad. Sci. USA (1988) 85:8805-8809), a partial cDNA sequence including fragments of 5′ coding sequence, (Cannon et al., Plant Molec. Biol. (1990) 15:39-47), or 3′ non-coding sequences (Ch'ng et al., Proc. Natl. Acad. Sci. USA (1989) 86:10006-10010). Cosuppression techniques may use the entire cDNA sequence (Napoli et al., supra; van der Krol et al., The Plant Cell (1990) 2:291-299), or a partial cDNA sequence (Smith et al., Mol. Gen. Genetics (1990) 224:477-481).

Standard molecular and genetic tests may be performed to further analyze the association between a gene and an observed phenotype. Exemplary techniques are described below.

The stage- and tissue-specific gene expression patterns in mutant versus wild-type lines may be determined, for instance, by in situ hybridization. Analysis of the methylation status of the gene, especially flanking regulatory regions, may be performed. Other suitable techniques include overexpression, ectopic expression, expression in other plant species and gene knock-out (reverse genetics, targeted knock-out, viral induced gene silencing [VIGS, see Baulcombe D, Arch Virol Suppl (1999) 15:189-201]).

Expression profiling, generally by microarray analysis, can be used to simultaneously measure differences or induced changes in the expression of many different genes. Techniques for microarray analysis are well known in the art (Schena M et al., Science (1995) 270:467-470; Baldwin D et al., (1999) Cur Opin Plant Biol. 2(2):96-103; Dangond F, Physiol Genomics (2000) 2:53-58; van Hal N L et al., J Biotechnol (2000) 78:271-280; Richmond T and Somerville S, Curr Opin Plant Biol (2000) 3:108-116). Expression profiling of individual tagged lines may be performed. Such analysis can identify other genes that are coordinately regulated as a consequence of the overexpression of the gene of interest, which may help to place an unknown gene in a particular pathway. For example, the analysis performed below demonstrates that under non-stress conditions an interfering RNA molecules suppresses NFY5A activity. Reducing the inhibition by the molecule can assist in conferring drought tolerance or optimizing expression or activation of NFY5A.

Analysis of gene products may include recombinant protein expression, immunolocalization, biochemical assays for catalytic or other activity, analysis of phosphorylation status, and analysis of interaction with other proteins via yeast two-hybrid assays.

Pathway analysis may include placing a gene or gene product within a particular biochemical, metabolic or signaling pathway based on a desired phenotype or by sequence homology with related genes. Alternatively, analysis may comprise genetic crosses with wild-type lines and other mutant lines (creating double mutants) to order the gene in a pathway, or determining the effect of a mutation on expression of downstream “reporter” genes in a pathway.

The disclosure further provides a method of identifying plants that have mutations in endogenous NFYA5 that confer increased drought tolerance, and generating drought-tolerant progeny of these plants that are not genetically modified. In one method, called “TILLING” (for targeting induced local lesions in genomes), mutations are induced in the seed of a plant of interest, for example, using EMS treatment. The resulting plants are grown and self-fertilized, and the progeny are used to prepare DNA samples. NFYA5-specific PCR is used to identify whether a mutated plant has a NFYA5 mutation. Plants having NFYA5 mutations may then be tested for drought tolerance; or alternatively, plants may be tested for drought tolerance, and then NFYA5-specific PCR is used to determine whether a plant having increased drought tolerance has a mutated NFYA5 gene. TILLING can identify mutations that may alter the expression of specific genes or the activity of proteins encoded by these genes (see Colbert et al (2001) Plant Physiol 126:480-484; McCallum et al (2000) Nature Biotechnology 18:455-457).

In another method, a candidate gene/Quantitative Trait Locus (QTLs) approach can be used in a marker-assisted breeding program to identify alleles of or mutations in the NFYA5 gene or orthologs of NFYA5 that may confer increased tolerance to drought (see Foolad et al., Theor Appl Genet. (2002) 104(6-7):945-958; Rothan et al., Theor Appl Genet (2002) 105(1):145-159); Dekkers and Hospital, Nat Rev Genet. (2002) January; 3(1):22-32). Thus, in a further aspect of the disclosure, a NFYA5 nucleic acid is used to identify whether a drought-tolerant plant has a mutation in endogenous NFYA5.

### EXAMPLES

Arabidopsis thaliana mutants, rdr2-1, dc12-1, dc13-1, and dc14-1, were obtained from Center for Gene Research and Biotechnology, Oregon State University. dcl1-7 and hen1-1 were provided by Xuemei Chen. rdr6 and sde4/nrpd1a were kindly provided by John Innes Center for Plant Science Research, Sainsbury Laboratory, United Kingdom. sgs3 was kindly provided by Laboratoire de Biologie Cellulaire, Institut National de la Recherche Agronomique, Versailles, France. hyl1 was a provided by The Huck Institute of Life Science, Pennsylvania State University. These mutants were in the Columbia (Col-0), Landsberg erecta (Ler), Noss-sen-0 (No), or C24 genetic backgrounds as indicated in the text and figures. The T-DNA insertion mutant of NFYA5 (SALK_{—}042760) was obtained from ABRC. Arabidopsis plants were grown under continuous light (70 μmol m−2 s−1) at 23±1° C. Nicotiana benthamiana plants were grown under 16 h light/8 h dark photoperiod at 23±1° C. For dehydration treatment, 2-week-old seedlings were pulled out of agar medium and left to dry on Whatman 3MM paper on laboratory bench for durations as indicated. For drought treatment, plants were grown in soil with sufficient water for 3 weeks, and then the water was withheld for certain periods of time.

Site-directed mutagenesis was performed to generate NFYA5 mutated in the region complementary to the small RNA by QuickChange II Site-Directed Mutagenesis Kit (Stratagene). This fragment was sequenced to ensure that only the desired mutations were introduced. NFYA5 with and without 3′-UTR was amplified with the primers indicated (with/without 3′-UTR forward 5′-CAC CAT GCA AGT CTT TCA AAG GAA AG-3′ (SEQ ID NO:9), with 3′-UTR reverse 5′-GTA ATG CAA TTG TAC TCT CGA G-3′ (SEQ ID NO:10) and without 3′-UTR reverse 5′-TCA AGT CCC TGA CAT GAG AGC TGA GG-3′ (SEQ ID NO:11)). Full length cDNA of At1g54150 was amplified with the following primers: forward 5′-CAC CAT GTC CTC GCC GGA GCG TGC TCT C-3′ (SEQ ID NO:12), reverse 5′-TAG TTC GAT CTC ACC GAG CTC CA-3′ (SEQ ID NO:13). These constructs were cloned into the plant expression GATEWAY destination vector pMDC32.

To generate pMDC32:miR169 constructs, a 200 by fragment surrounding the miRNA sequence including the fold-back structure was amplified from genomic DNA with the following primers: miR169a forward 5′-CAC CTG GGT ATA GCT AGT GAA ACG CG-3′ (SEQ ID NO:14) and reverse 5′-CCT TAG CTT GAG TTC TTG CGA-3′ (SEQ ID NO:15), miR169b forward 5′-CAC CCC CAA CGG AGT AGA ATT G-3′ (SEQ ID NO:16) and reverse 5′-CTC ATA CGG TCG ATG TAA TCC GT-3′ (SEQ ID NO:17), miR169c forward 5′-CAC CTC GTC CAT TAT GAG TAT T-3′ (SEQ ID NO:18) and reverse 5′-CTA ATA TGA TAT GAA TAT GGA TGA-3′ (SEQ ID NO:19), miR169h forward 5′-CAC CTC ATA TAA GAG AAA ATG GTG-3′ (SEQ ID NO:20) and reverse 5′-CCA AAA AAG AGA AAT GTG AAT GAG-3′ (SEQ ID NO:21). The amplified fragments were introduced into the pENTRTM/D-TOPO vector (Invitrogen) and cloned into pMDC32 by LR reactions (Invitrogen).

For NFYA5 promoter:: GUS construct, a 1.7 kb fragment upstream from the initiation codon was amplified with the forward primer 5′-CAC CTG TAT GAC ATA TTC TGT GTG GAG-3′ (SEQ ID NO:22) and reverse primer 5′-TGC AAA TTG GGT ATT GGC TAT G-3′ (SEQ ID NO:23) and cloned into the pMDC164 vector following Gateway recombination.

A fusion of YFP to the C-terminal end of NFYA5 was generated and introduced to pEarleyGate 101 vector by Gateway recombination. YFP images were collected on a Leica SP2 confocal microscope.

Total RNA was extracted from wild type, mutants and transgenic plants with Trizol reagent (Invitrogen). For enrichment of small RNAs, high molecular weight RNA was selectively precipitated by the addition of one volume of 20% PEG-1M NaCl (Llave et al., 2002). High molecular weight RNA was separated on 1.2% formaldehyde-MOPS agarose and low molecular weight RNA was fractioned on 17% denaturing polyacrylamide gels. The blots were probed and washed as described (Borsani et al., 2005).

For real-time RT-PCR, first-strand cDNA was synthesized using SuperScript™ III First-Strand Synthesis Supermix (Invitrogen). Primers specific for precursor of miR169 were used to detect expression levels of miR169 (See Table 1). Primers were also designed to detect the transcription level of NFYA5. Real-time RT-PCR was carried out in iQ5 Real-Time PCR Detection System using iQ™ SYBR Green Supermix (Bio-Rad). Each experiment was replicated three times. The comparative Ct method was applied.

TABLE 1

| Primer Name (SEQ ID NO: ) | Sequence (5′-3′) |
|---|---|
| miR169a Forward (24) | TGGGTATAGCTAGTGAAACGCG |
| miR169a Reverse (25) | CCTTAGCTTGAGTTCTTGCGA |
| miR169b Forward (26) | CTCCACTCCCTAAACCATCACAAC |
| miR169b Reverse (27) | CTCATACGGTCGATGTTAATCCGT |
| miR169c Forward (28) | CTACATTCACAAACGAGAGAATT |
| miR169c Reverse (29) | GCTCTACTTTAGCATCTCAACCA |
| miR169h Forward (30) | GTAGTCTTGTGGGATACTCATCA |
| miR169h Reverse (31) | ATCCCCAAATTTGGAGGCCAAACA |
| miR169i Forward (32) | AAGGTGTCTCCTGGGTTGAAAAT |
| miR169i Reverse (33) | CCATGATCATTCATGTCGTGCCT |
| miR169j Forward (34) | GAGAGCACATCCATGTGAGGAA |
| miR169j Reverse (35) | GTCAGGCAAGTCATCCTTGGCT |
| miR169k Forward (36) | CTCTTTGGCTATCTTGACATGCT |
| miR169k Reverse (37) | CCAAGGAGACTGCCTGATGTCT |
| miR169l Forward (38) | GAGAGCACATGAATGTAAGGCA |
| miR169l Reverse (39) | AATAATGTGTAGACTCAGCCACAT |
| miR169l Reverse (40) | CTTGCGGGTTAGGTTTCAGGCA |
| miR169m Reverse (41) | GCCTCGAAATCATGAACATTATCT |
| miR169n Forward (42) | AGAGAGCACATGCATGTATGGA |
| miR169n Reverse (43) | GAAAAGTAGGTATAACATGGATGG |
| NFYA5 Forward (44) | GAAGATTCATCTTGGGGAAACTC |
| NFYA5 Reverse (45) | GAGCAGGAAACACAGAGTCTTGA |
| At4g15120 Forward (46) | GGTTGGGTGTTTCCCGGCATCGGA |
| At4g15120 Reverse (47) | GGCTTCTCCTAAGATCTGATCTCCA |
| At1g17170 Forward (48) | CCTTCCCTCCGATCCTTACAAGA |
| At1g17170 Reverse (49) | CAACCCAAGTTTCTTCCTACGTTCG |
| At2g37870 Forward (50) | CGTTGAGGCGGCAGGTGAGTGT |
| At2g37870 Reverse (51) | TGTAACGTCCACATCGCTTGCCA |
| At2g42530 Forward (52) | TCAGTGGCATGGGTTCTTCTTCCA |
| At2g42530 Reverse (53) | GAGGTCATCGAGGATGTTGCCGT |
| At2g42540 Forward (54) | GTTCTCACTGGTATGGCTTCTT |
| At2g42540 Reverse (55) | GTCTTTCGCTTTCTCACCATCTGCT |
| 18S Forward (tobaco) (56) | AGGAATTGACGGAAGGGCA |
| 18S Reverse (tobaco) (57) | GTGCGGCCCAGAACATCTAAG |
| Tub8 Forward (58) | ATAACCGTTTCAAATTCTCTCTCTC |
| Tub8 Reverse (59) | TGCAAATCGTTCTCTCCTTG |

Site-directed mutagenesis construct with and without 3′-UTR constructs were transformed into Agrobacterium tumefaciens strain 3301. Overnight cultures were harvested and mixed at 1:1 ratio with various combinations. After 1 h incubation at room temperature in 10 mM MgCl2, 10 mM MES (pH 5.6) and 150 μM acetosyringone, Agrobacterium suspension was coinfiltrated into 3-week-old N benthamiana leaves. Leaves were harvested 2 days after the infiltration and small RNA extraction and blotting performed as described above.

Rosette leaves from 3-week-old soil-grown plants at similar developmental stages were harvested. Leaves were frozen immediately in liquid nitrogen and observed for guard cells by environmental scanning electron microscopy (HITACHI, TM 1000). Width and length of stomotal pores were measured for statistical analysis.

For water loss measurement, six leaves per individual of mutant and wild type plants growing under normal conditions for 3 weeks were excised and fresh weight was determined at designated time intervals. Four replicates were done for each line. Water loss was represented as the percentage of initial fresh weight at each time point.

Anthocyanin contents were measured as described by Rabino and Mancinelli (1986) and Sukar et al. (2006). The pigments were extracted with 99:1 methanol:HCl (v/v) at 4° C. and OD₅₃₀ and OD₆₅₇ for each sample were measured and OD₅₃₀−0.25×OD₆₅₇ was used to compensate for the contribution of Ch1 and its products to the absorption at 530.

Histochemical localization of GUS staining was carried out by incubating the transgenic plants in 1 mg mL 15-bromo-4-chloro-3 indolyl β-D-glucuronic acid, 0.1 M Na₂HPO₄ buffer (pH 7.0), 0.5 mM K₃(Fe[CN]₆), and 10 mM EDTA overnight at 37° C., followed by clearing with 70% ethanol.

GUS activity was assayed according to the procedure of Jefferson (1987). 100 mg frozen tissues were homogenized in 100 μl extraction buffer (50 mM NaPO₄, pH 7.0, 1 mM Na₂EDTA, 0.1% (v/v) Triton X-100, 0.1% [w/v] sodium lauryl sarcosine and 10 mM dithiontheitol), and centrifuged for 10 min at 4° C. at 13,000 rpm. The fluorogenic assay was incubated in a 0.5-mL volume extraction buffer supplied with 1 mM 4-methylumbelliferyl-β-D-glucuronide (Sigma) for 2 h, then stopped by 0.2 M Na₂CO₃. Protein concentration was determined according to the Bio-Rad protocol provided with the protein assay kit. GUS activity was calculated as picomoles MU per minute per milligram of protein.

For Affymetrix GeneChip array analysis, wild-type and 35S::NFYA5 seedlings were grown on MS plates for 15 days at 22° C. with a cycle of 16 h light and 8 h darkness. Total RNA was extracted by using RNeasy Plant Mini Kit (Qiagen) and used for preparation of biotin-labeled complementary RNA targets. Microarray analysis was performed as described by Breitling et al (2004). Up- and down-regulated gene list using the Bioconductor package, RankProd (Gentleman et al., 2004, Hong et al., 2006) were generated. For predicting consensus novel cis-regulatory element, the AlignACE program (Hughes et al., 2000) was used. The program was applied to 1 kb upstream promoter sequences of up- or down-regulated genes. Out of several candidate consensus elements, one was selected that contains a weak CCAAT consensus motif which was found within promoters of up-regulated genes.

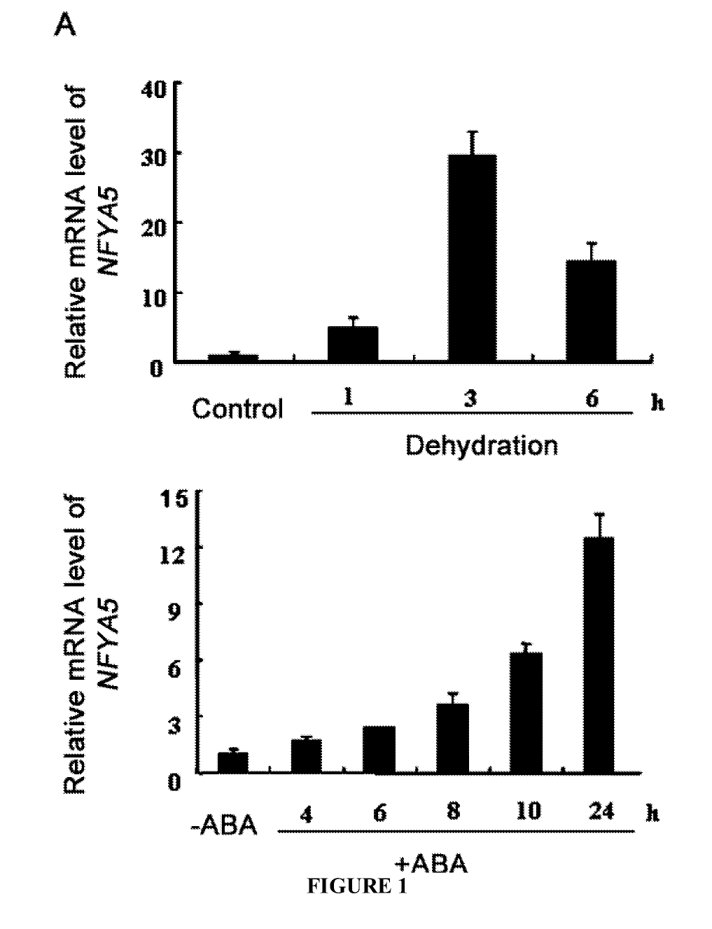

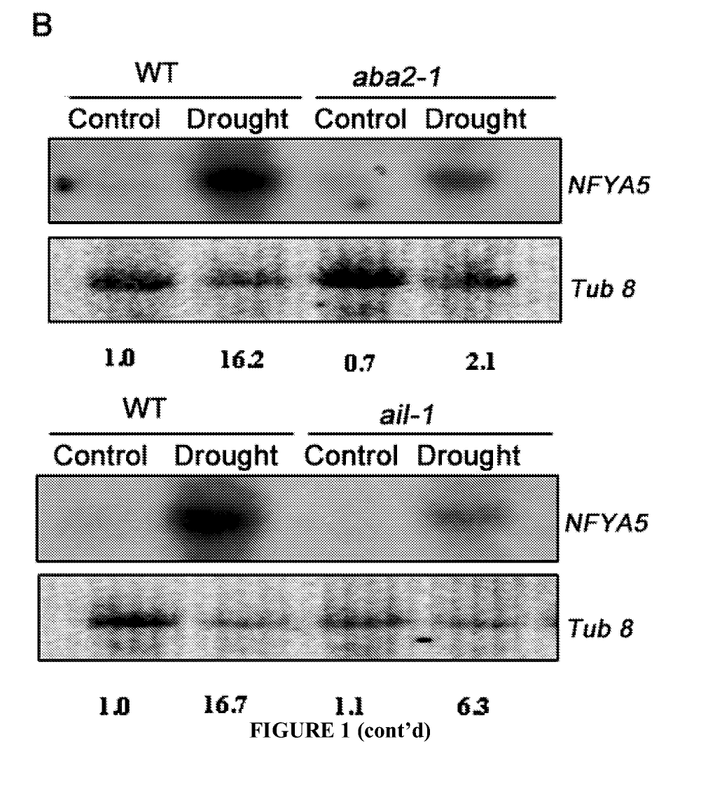

NFYA5 was initially investigated because it is annotated to overlap with another gene (At1g54150) on the antisense strand in their 3′ UTR regions to form a natural cis-antisense transcript (NAT) gene pair. The transcript of NFYA5 accumulated rapidly in response to dehydration treatment and showed a peak induction of approximately 30-fold at 3 h after the start of dehydration treatment (FIG. 1A). When the dehydration treatment was continued for longer periods, the mRNA level of NFYA5 decreased but was still higher than that of control (FIG. 1A). The transcript of NFYA5 was also highly induced in soil grown plants subjected to drought stress (FIG. 1B).

ABA accumulation is required for some drought stress-induced up-regulation of gene expression (Shinozaki and Yamaguchi-Shinozaki, 1996; Zhu, 2002). Thus, the response of NFYA5 to ABA treatment was examined NFYA5 expression increased approximately 13-fold by 24 h after the application of 100 μM ABA. To verify that ABA is associated with drought-induced increase of NFYA5 expression, an ABA-deficient (aba2-1) mutant and an ABA-insensitive (abi1-1) mutant was generated. In both the Col-0 and Ler wild types, the expression of NFYA5 was strongly induced by withholding watering for 10 days; however, drought-induced accumulation of NFYA5 mRNA was substantially reduced in aba2-1 and abi1-1 (FIG. 1B). This result suggests that AtNFYA5 expression is at least partly dependent on ABA signaling.

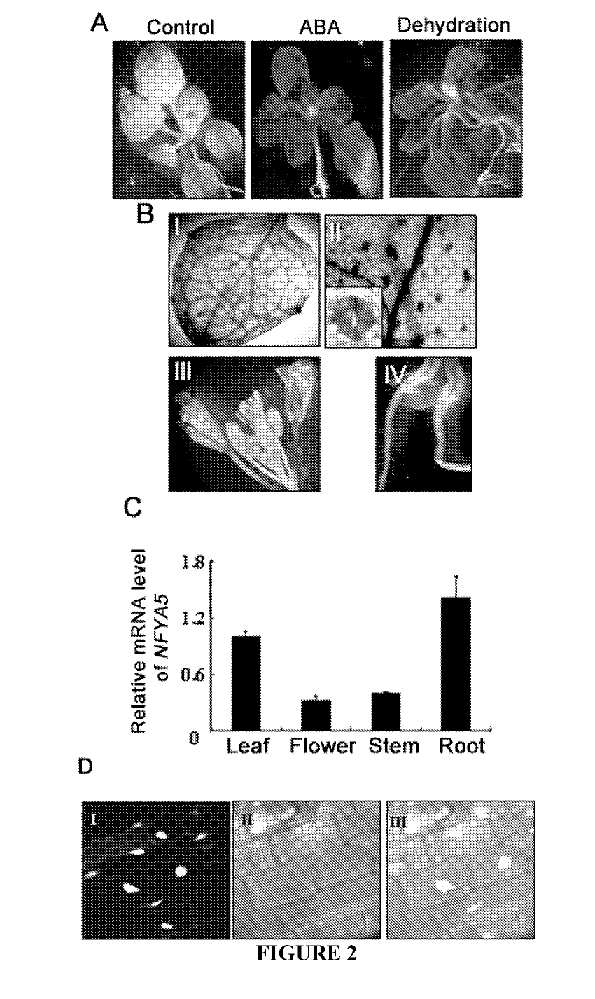

Although the real-time PCR and RNA blot assays both showed a clear induction of NFYA5 RNA accumulation in response to dehydration and drought stress or ABA, such assays could not address whether this increase was caused by increased promoter activity or altered RNA stability. To address this question, a promoter::GUS fusion using a 1.7 kb fragment upstream from the initiation codon of the NFYA5 was constructed. Analysis of GUS staining patterns in several transgenic lines showed that GUS staining increased in response to dehydration stress or ABA treatment (FIG. 2A). Thus, these treatments did increase the transcriptional activity of the NFYA5 promoter. However, quantitative analysis indicated that GUS activity only increased approximately 5-fold in response to dehydration treatment. This was much less than the 30-fold upregulation of NFYA5 mRNA level found by quantitative PCR analysis (FIG. 1A). Thus, in addition to transcriptional induction, post transcriptional regulation of NFYA5 also plays a role.

The promoter::GUS transgenic lines were also used to examine the expression pattern of NFYA5. NFYA5 expression was high in leaf tissues with prominent expression in both the leaf vascular system and, importantly, a high level of expression in guard cells (FIG. 2B, panels I and II). GUS staining was also observed in floral tissues and the root vascular system (FIG. 2B, panels III and IV). Quantitative PCR analysis of NFYA5 mRNA levels in different tissues was consistent with the tissue pattern of GUS staining and showed that expression was highest in leaf and root tissues with significant expression also occurring in floral and stem tissues (FIG. 2C).

In Arabidopsis, the A subunit of the NF-Y complex is encoded by a 10-member gene family. Though other regions of the proteins vary, the NF-YAs contain a highly conserved core region that consists of two functional subdomains: an NF-YB/NF-YC binding subdomain and a DNA binding subdomain which are connected by a small linker (Romier et al., 2003; Wenkel et al., 2006). The Psort II program predicted a nuclear localization of NFYA5 protein with 70% certainty. To confirm the subcellular localization of NFYA5 protein, a translational fusion between yellow fluorescence protein (YFP) and the C terminus of NFYA5 was transformed into Arabidopsis. Cells expressing the NFYA5-YFP fusion protein showed that the YFP signal only appeared in the nucleus (FIG. 2D).

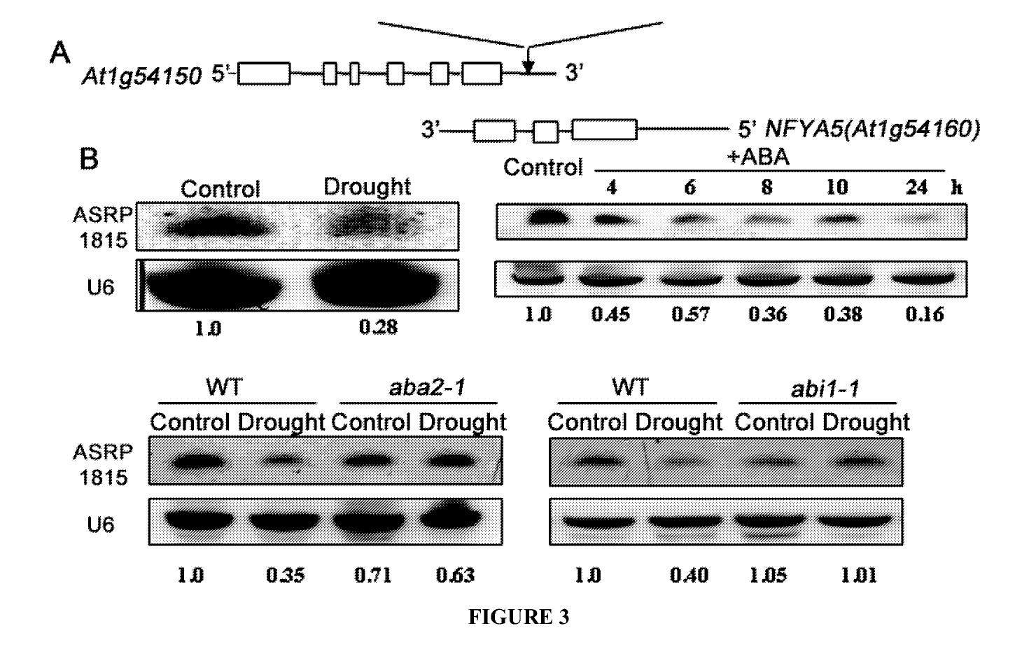

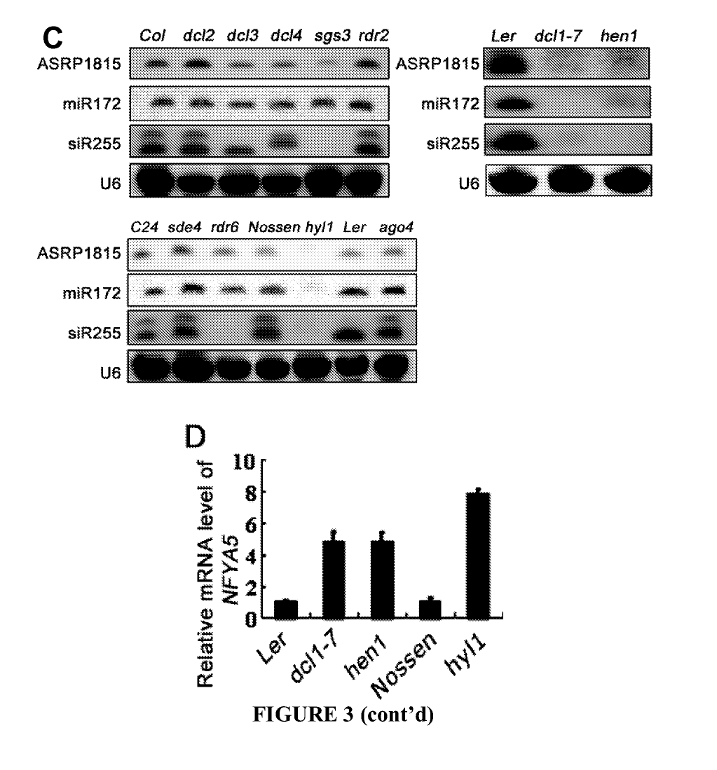

The high level of NFYA5 mRNA accumulation under dehydration which could not be explained solely by the promoter activity of AtNFYA5 strongly suggested the presence of another regulatory mechanism operating at the post-transcriptional level. One possibility is regulation by small RNA(s). To begin to investigate this possibility, the Arabidopsis MPSS Plus Database (world wide web at mpss.ude1.edu/at/) was search and two small RNA signatures (17 nt) that matched the 3′ end of At1g54150 and are complementary to the 3′ UTR of AtNFYA5 were identified. The small RNA signatures in the Arabidopsis MPSS Plus Database was identical to part of ASRP1815 (19 nt) in the ASRP small RNA database (world wide web at asrp.cgrb.oregonstate.edu). Thus an oligonucleotide probe was designed complementary to ASRP 1815 (FIG. 3A).

Using this oligonucleotide probe, a 21-nt small RNA in plants grown under normal growth conditions was detected. In plants subjected to water withholding for 10 days, the level of the ASRP1815 small RNA decreased to ˜28% of the level in unstressed plants (FIG. 3B). In this same treatment, the expression of NFYA5 increased 14-fold in response to the drought stress. This is consistent with the possibility that reduced expression of ASRP1815 under drought may reduce the small RNA-directed degradation of NFYA5 mRNA. Also consistent with ABA induced NFYA5 mRNA accumulation, ABA treatment suppressed the level of ASRP1815 to approximately 16% of the control level (FIG. 3B). In both the Col-0 and Ler wild types, the expression of ASRP 1815 was strongly suppressed by drought stress; however, ASRP 1815 expression in the aba2-1 and abi1-1 mutants was not substantially affected by drought (FIG. 3B). Therefore, drought stress suppression of ASRP 1815 was dependent on ABA signaling.

The fact that NFYA5 and At1g54150 form a NAT gene pair raised the possibility that they may generate a nat-siRNA which regulates NFYA5. The biogenesis of nat-siRNAs requires DCL2 or DCL1, RDR6, SGS3 and NRPD1a (Borsani et al., 2005; Katiyar-Agarwal et al., 2006). To test if ASRP1815 was a nat-siRNA, its biogenesis in mutants defective in various proteins known to be required for biogenesis of specific types of small RNAs was examined ASRP1815 was still produced in dc13, rdr2, dc14, dc12, rdr6, sgs3 or nrpd1a (FIG. 3C). This suggested that it was not a hereochromatic siRNA, tasiRNA or nat-siRNA of the types described previously. Instead, ASRP1815 was absent in hen1, dcl1-7 and hyl1 (FIG. 3C). The requirement of these components suggests that ASRP1815 is probably a microRNA. Disruption of NFYA5 expression had little effect on ASRP1815 accumulation, consistent with the notion that ASRP1815 is not a nat-siRNA.

The levels of NFYA5 mRNA in the mutants where the ASRP 1815 small RNA was absent were then examined. The level of NFYA5 mRNA was higher in hen1, dc11-7 and hyl1 than that in the wild type (FIG. 3D). This result suggested ASRP1815 indeed down-regulates NFYA5 expression.

The requirement of HEN1, DCL1 and HYL1 suggested that a miRNA was being detected with the ASRP1815 probe. Thus a search was performed for sequences homologous to ASRP1815 in small RNA databases and found that ASRP1815 is homologous to sequences of the miR169 (21 nt) family. The family of miR169 in Arabidopsis contains 14 members and this miRNA family is conserved in Oryza sativa and Populus trichocarpa (Bonnet et al., 2004; Jones-Rhoades and Bartel, 2004; Sunkar and Zhu, 2004; Sunkar et al., 2005). Based on sequence of the miRNA produced, the MIR169a/b/c//h/i/j/k/l/m/n family can be divided into three subgroups. MIR169a represents the first sub-group, MIR169b and MIR169c form the second group and the third group is made up of MIR169h/i/j/k/l/m/n. The main difference among the subgroups is the sequence at the 3′ end: the last two nucleotides of MIR169a are G and A, while those of MIR169b/c and MIR169h/i/j/k/l/m/n are GG and UG respectively. Also, the 5′ end of MIR169h/i/j/k/l/m/n is UA, which is different from CA of other two subgroups.

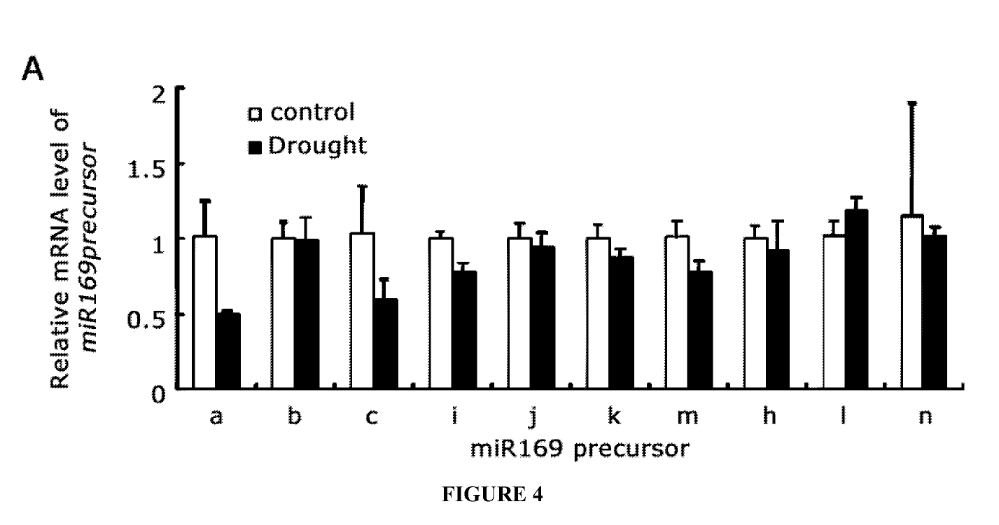

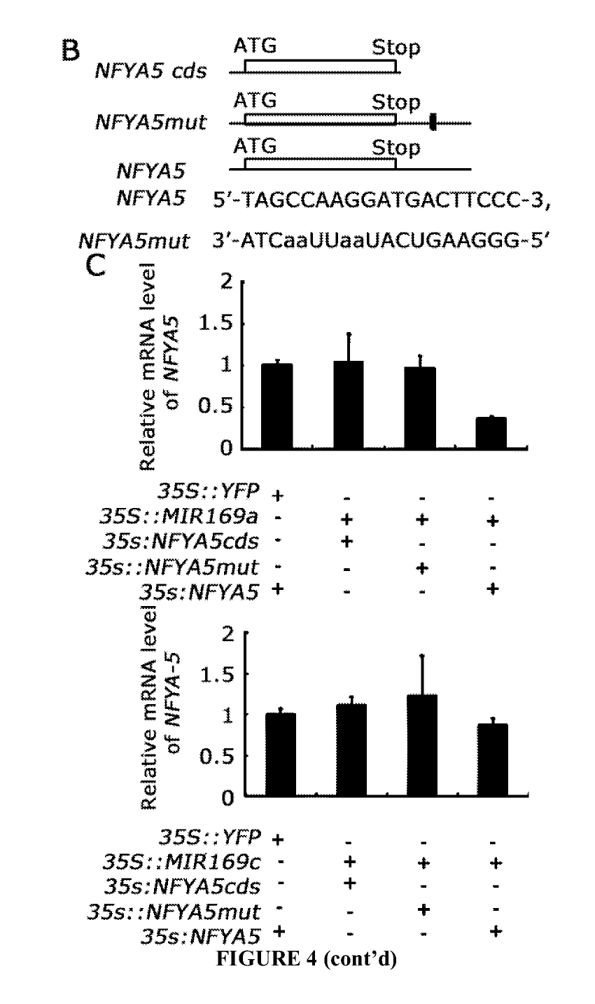

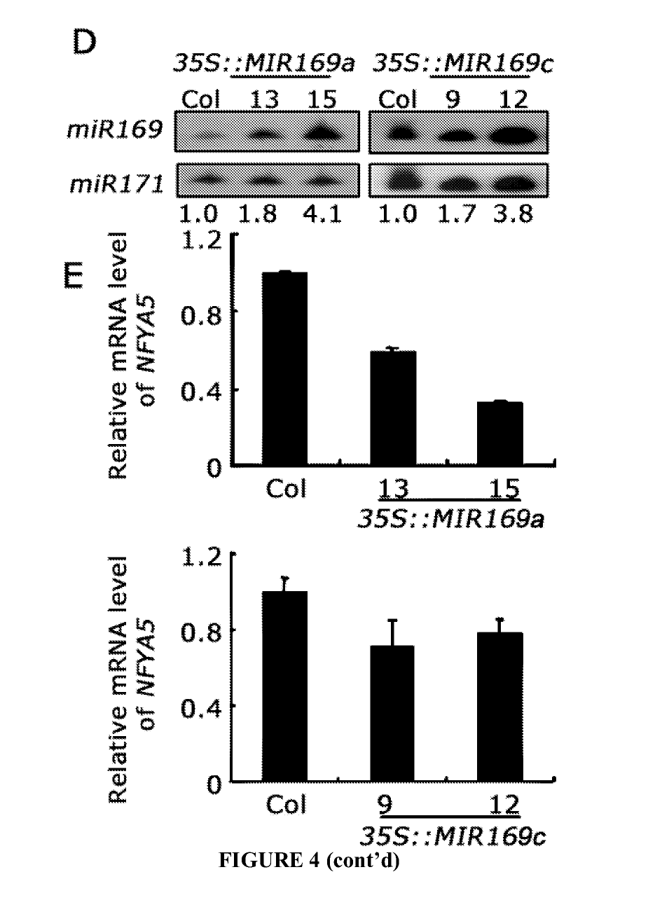

Because of their sequence similarity, the MIR169 family members cannot be differentiated in small RNA blots because of cross-hybridization. To determine which of the MIR169 loci may regulate NFYA5, a real-time RT-PCR was carried out using miR169 locus-specific primers to determine if expression of any of the miR169 loci is regulated by drought stress. RNA was extracted from soil-grown Arabidopsis plants that had been subjected to water withholding for 10 days. Only MIR169a and MIR169c exhibited substantial changes in transcript abundance in response to drought stress (FIG. 4A). Both MIR169a and MIR169c were downregulated by drought stress, consistent with the down-regulation of the mature miRNA by drought. Further experiments were conducted to determine if MIR169a and MIR169c could down-regulate AtNFYA5 mRNA.

It has, in many cases, been assumed that members of a miRNA family have mostly redundant functions; however, Sieber et al. (2007) recently provided evidence that closely related miRNAs that were predicted to target the same genes had in fact different functions during development. To test whether MIR169a or MIR169c may have a specific role in regulating NFYA5 expression, transient coexpression assays in were performed in Nicotiana benthamiana. Because the target site of miR169 is located in the 3′ UTR of NFYA5, three NFYA5 constructs were tested: a full length NFYA5 including the 3′ UTR, a construct without 3′ UTR and another construct that was mutated in the 3′ UTR to introduce four mismatches between it and miR169 (FIG. 4B). Both the NFYA5 and miR169a or miR169c constructs were expressed under control of the 35S promoter. After 2 days of coexpression in N benthamiana, RNA was extracted and NFYA5 expression analyzed by quantitative RT-PCR. mRNA levels from the constructs which lacked a functional miR169 target site, NFYA5cds and NFYA5mut, were not affected by coexpression with miR169 (FIG. 4C). However, the level of NFYA5 mRNA was decreased significantly (37% of the control level) when coexpressed with MIR169a (FIG. 4C). Interestingly, coexpression with miR169c caused only a 13% decrease in the level of NFYA5 transcript. The results suggested that the degradation of NFYA5 mRNA was mainly directed by MIR169a. Small RNA blots prepared from the same samples and probed with oligonucleotides complementary to MIR169 clearly showed that a 21-nt small RNA was highly expressed in all of the co-expression samples. Thus, the failure of strong miR169c-directed cleavage of AtNFYA5 was not due to a lack of miR169c expression.

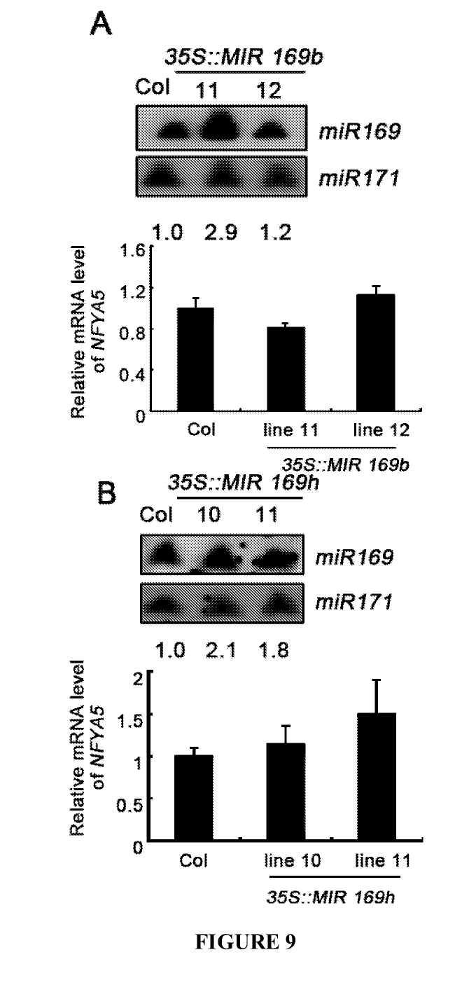

To confirm these results, the precursors of MIR169a and MIR169c was overexpressed in Arabidopsis and chose lines with similar miR169 expression levels to quantify the effect of miR169a and c overexpression on NFYA5 (FIG. 4D). In agreement with the transient coexpression assay results, overexpression of miR169a caused a larger decrease in the level of NFYA5 mRNA than overexpression of miR169c (FIG. 4E). Two other members of the miR169 family were also chosen, miR169b and h, and they were overexpressed in Arabidopsis. The relative mRNA levels of NFYA5 did not change substantially despite the overexpression of the miRNA (FIG. 9). These results again suggest MIR169a as the major miRNA locus important for the regulation of AtNFYA5 expression.

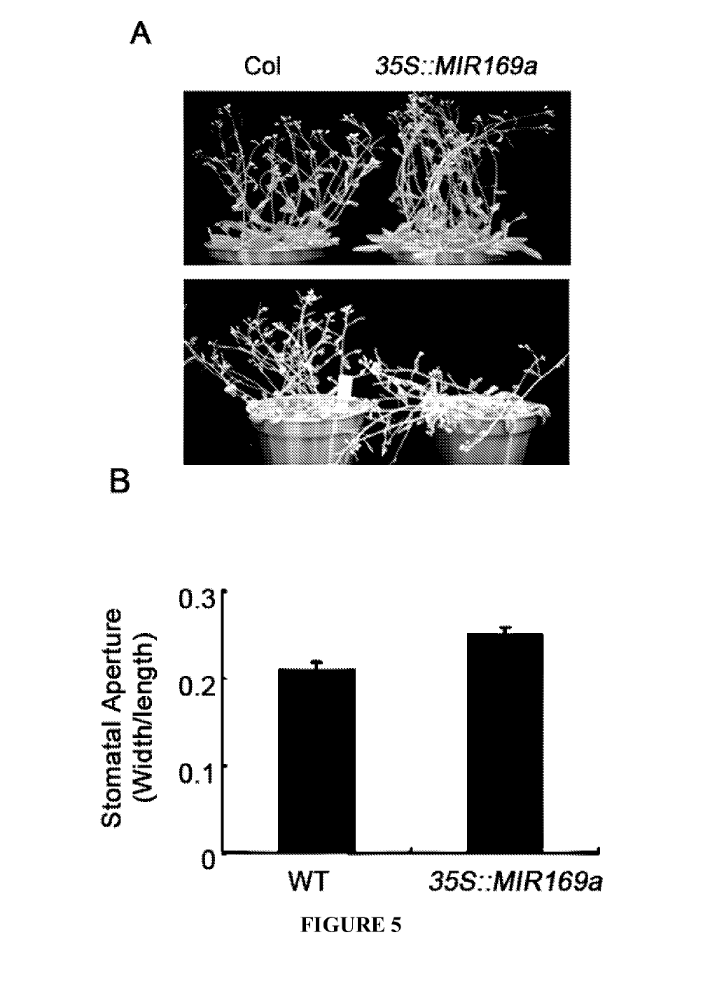

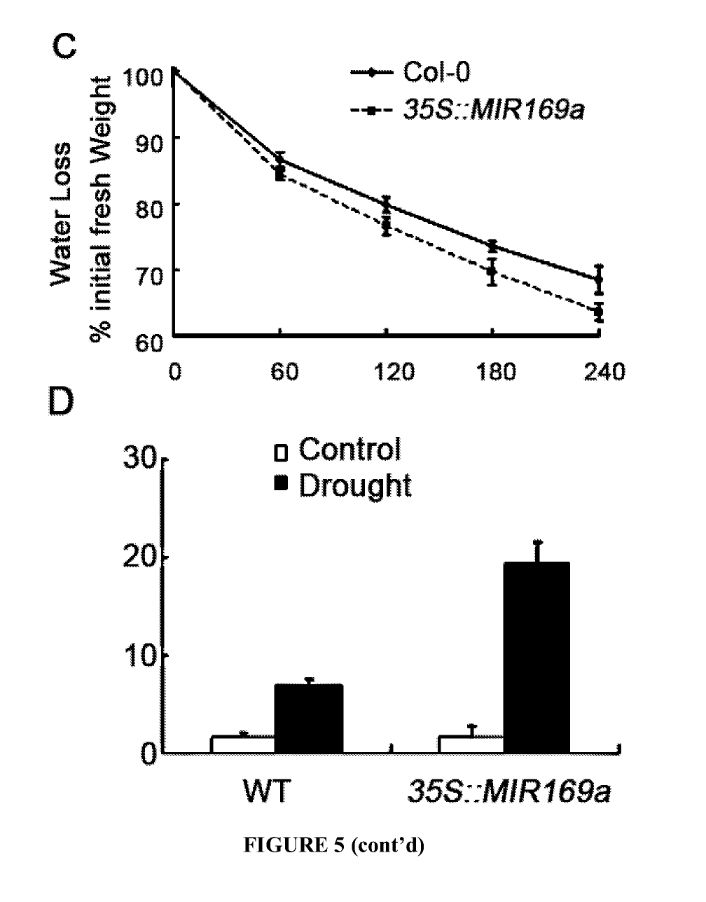

Drought responsive gene regulation and ABA signaling are crucial for drought resistance (Pei et al., 1998; Zhu, 2002). The drought- and ABA-inducible expression of NFYA5 and its strong expression in guard cells prompted us to analyze its potential role in drought resistance. Firstly, plants overexpressing miR169a (35S::MIR169a, line #15), in which the level of NFYA5 mRNA was ˜33% of the wild type were tested (FIG. 4D). Wild-type and 35S::MIR169a—15 plants were grown for 3 weeks in soil and were then subjected to water withholding for 8 days. 35S::MIR169a-15 plants showed leaf rolling and the leaves became purple whereas the wild type plants were still turgid and their leaves remained green (FIG. 5A). The result suggested that 35S::MIR169a-15 plants may have depleted the soil water more rapidly than the wild type and thus wilted more quickly. To investigate this possibility, leaves from 35S::MIR169a-15 and WT plants grown in soil were analyzed to determine their stomatal apertures. The stomatal aperture index of 35S::MIR169a-15 leaves was 0.25, which was nearly 20% greater than that of WT (FIG. 5B). Consistent with these results, detached leaves of 35S::MIR169a-15 plants consistently lost water more quickly than those of the wild type (FIG. 5C), suggesting that the more rapid appearance of wilting after water withholding in 35S::MIR169a-15 could be attributed at least in part to an inability of these plants to efficiently close their stomata and reduce transpiration. Because ABA is a regulator of stomatal aperture and transpiration, this phenotype is consistent with the ABA-induced expression of NFYA5 and suggests that NFYA5 may be important for ABA signaling in guard cells. Another indicator of stress sensitivity is the accumulation of the purple flavonoid pigment, anthocyanin, in leaves. The anthocyanin levels in 35S::MIR169a-15 plants after withholding water for 8 days was 19.41 μg g−1FW, which was about 3 times as much as that of the wild type, again supporting that 35S::MIR169a-15 was more sensitive to drought stress. The results suggest that adequate expression of NFYA5 is required for drought resistance.

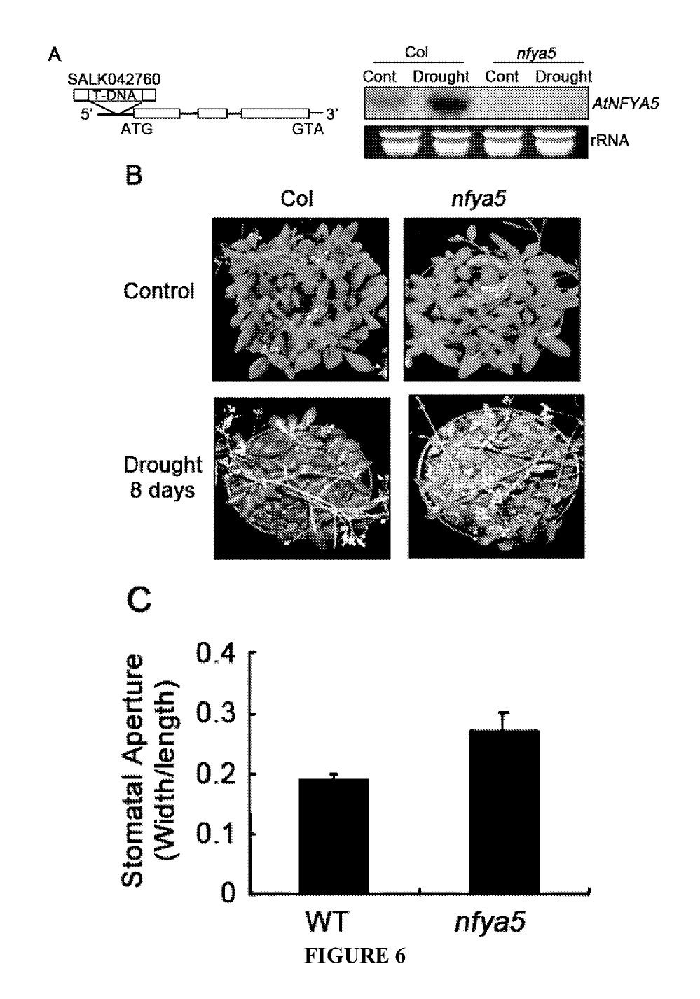

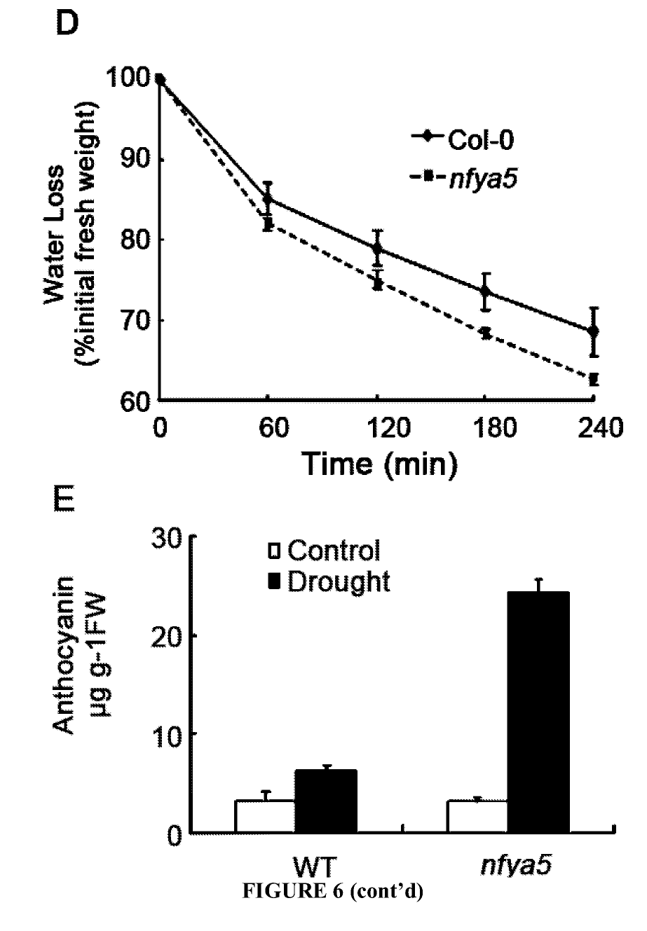

The publicly available T-DNA collections were searched and obtained a T-DNA insertion mutant (SALK_{—}042760 in the Columbia background) from the Arabidopsis Biological Resource Center to further investigate the function of NFYA5. Plants homozygous for the T-DNA insertion were identified by PCR and sequencing of the T-DNA flanking region confirmed the insertion site in the promoter region of NFYA5 (FIG. 6A). RNA blot analysis showed that the NFYA5 transcript was absent in the T-DNA line designated as nfya5 (FIG. 6A). In agreement with the phenotypes of 35S::MIR169a-15 plants, nfya5 knock-out mutant plants were also hypersensitive to drought stress (FIG. 6B). The stomatal aperture index of nfya5 leaves was 0.27, which was 42% greater than that of wild type leaves (FIG. 6C). Consistent with these results, detached leaves of nfya5 lost water more quickly than those of wild type leaves (FIG. 6D). The anthocyanin levels in nfya5 leaves after withholding water for 8 days was 4 times as much as that of the wild type, again supporting that nfya5 plants were more sensitive to drought stress. These results show that NFYA5 is a factor for drought resistance.

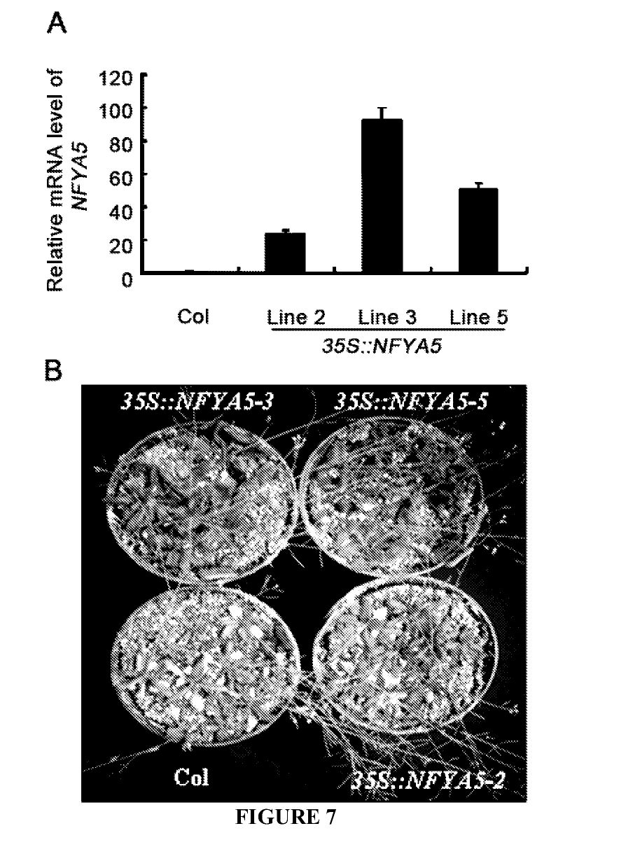

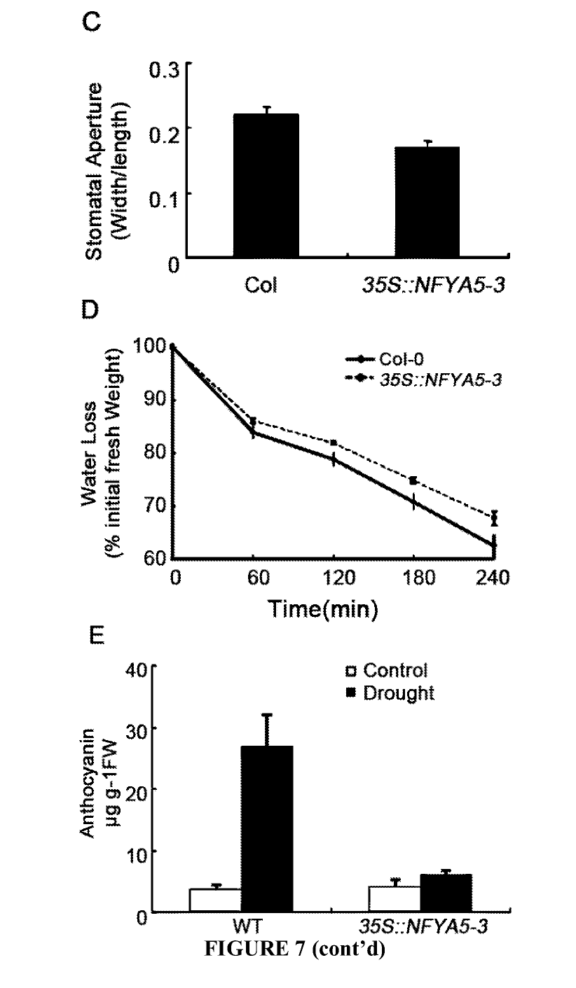

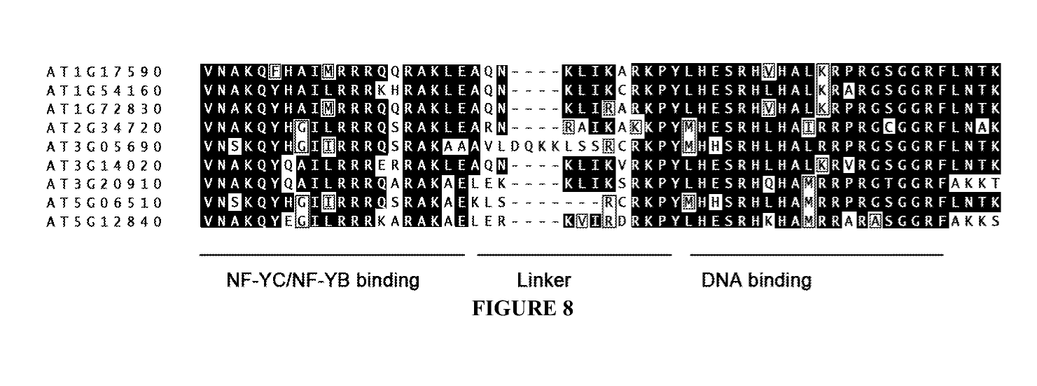

To further characterize the function of NFYA-5 in drought resistance, a transgenic Arabidopsis plants overexpressing the gene under the constitutive CaMV 35S promoter were generated. Three transgenic lines (#2, 3 and 5) were chosen for further analysis based on their high level of NFYA5 expression (FIG. 7A). To evaluate the effects of NFYA5 overexpression, 3-week-old soil-grown wild type and 35S::NFYA5 plants were subjected to water withholding for 14 days. At the fourteenth day of water withholding, most of the wild type plants appeared dehydrated, but the 35S::NFYA5 plants appeared less dehydrated than the wild type (FIG. 7B). In contrast to nfya5, the stomatal aperture of 35S::NFYA5-3 was smaller than that of wild type (FIG. 7C). Detached leaves of 35S::AtNFYA5-3 lost water more slowly than those of wild type (FIG. 7D), and the anthocyanin levels in 35S::NFYA5-3 after withholding water for 14 days were much lower (FIG. 6E). These results show that overexpression of NFYA5 improves plant drought resistance.

The nuclear localization and DNA binding domain of NFYA5 suggest that the protein may act in regulating the expression of other genes important for drought resistance. To test this possibility, microarray experiment were performed by using Affymemetrix Arabidopsis ATH1 Genechips. A total of 28 genes showed a 2-fold or more change in expression in 35S::NFYA5 compared to wild type seedlings under non-stress conditions, in which 17 and 11 genes were increased or decreased, respectively, in the transgenic plants (Table 2). Most of these genes have known or presumed function associated with abiotic stress responses, and a number of them appear to be involved in oxidative stress (e.g. subunit of cytochrome b6-f complex, GST, peroxidases and oxidoreductase family protein). Out of these genes affected by NFYA5 ectopic expression, most of them contain the CCAAT motif in their promoter regions (Table 2) as expected since this short sequence motif can be found in a substantial fraction of gene promoters in general. Using the AlignACE program (Hughes et al., 2000), a consensus cis-regulatory element, TX(C/A)TTXGX(C/A)CAXT (SEQ ID NO:60), that contains the “CCAAT motif” in the promoters of a subset of the genes was identified that showed increased expression in the NFYA5 overexpression plants (Table 2). It is possible that these genes are the direct targets of NFYA5.

TABLE 2
List of genes with expression changes of at least 2-fold (P < 0.05) in the
35S::NFYA5 transgenic plants from microarray analysis.

| | | | Number of | Number of | Change |
|---|---|---|---|---|---|
| | | | CCAAT | novel | Fold |
| Affy ID | Gene ID | Description | motif | element | (OX/WT) |
| 263158_at | AT1G54160 | NFYA5 | 0 | 0 | 12.72 |
| 245275_at | AT4G15210 | Cytosolic beta-amylase expressed | 1 | 0 | 3.34 |
| | | in rosette leaves and inducible by | | | |
| | | sugar | | | |
| 244966_at | ATCG00600 | Cytochrome b6-f complex, subunit V | 2 | 1 | 3.33 |
| 262517_at | AT1G17180 | Glutathione transferase belonging | 4 | 0 | 3.24 |
| | | to the tau class of GSTs | | | |
| 267565_at | AT2G30750 | Putative cytochrome P450 | 0 | 0 | 3.08 |
| 261021_at | AT1G26380 | FAD-binding domain-containing | 1 | 0 | 3.02 |
| | | protein | | | |
| 266098_at | AT2G37870 | Protease inhibitor/seed storage/ | 2 | 1 | 2.81 |
| | | lipid transfer protein family | | | |
| | | protein | | | |
| 260568_at | AT2G43570 | Chitinase, putative | 5 | 1 | 2.77 |
| 262518_at | AT1G17170 | Glutathione transferase belonging | 1 | 2 | 2.46 |
| | | to the tau class of GSTs | | | |
| 247224_at | AT5G65080 | MADS-domain protein | 5 | 1 | 2.38 |
| 267101_at | AT2G41480 | Peroxidase | 0 | 0 | 2.29 |
| 244993_s_at | ATCG01000 | [ATCG01000]hypothetical protein | 44 | 0 | 2.28 |
| | ATCG01130 | [ATCG01130]hypothetical protein | | | |
| 249481_at | AT5G38900 | DSBA oxidoreductase family | 3 | 1 | 2.23 |
| | | protein | | | |
| 263497_at | AT2G42540 | COR15A | 1 | 0 | 2.20 |
| 263495_at | AT2G42530 | Cold-responsive protein | 7 | 0 | 2.14 |
| 247718_at | AT5G59310 | Member of the lipid transfer | 3 | 0 | 2.01 |
| | | protein family | | | |
| 254232_at | AT4G23600 | CORI3 | 4 | 0 | 2.00 |
| 246375_at | AT1G51830 | ATP binding/kinase/protein | 2 | 0 | 0.50 |
| | | serine/threonine kinase | | | |
| 251226_at | AT3G62680 | PRP3; structural constituent of cell | 2 | 0 | 0.48 |
| | | wall | | | |
| 254828_at | AT4G12550 | AIR1; lipid binding | 1 | 0 | 0.48 |
| 264577_at | AT1G05260 | RCI3); peroxidase | 1 | 0 | 0.47 |
| 258338_at | AT3G16150 | L-asparaginase, putative/L- | 3 | 0 | 0.47 |
| | | asparagine amidohydrolase, | | | |
| | | putative | | | |
| 254044_at | AT4G25820 | XTR9 | 2 | 0 | 0.46 |
| 266353_at | AT2G01520 | Major latex protein-related | 3 | 0 | 0.46 |
| 258473_s_at | AT3G02620, | [AT3G02620] acyl-desaturase, | 31 | 0 | 0.45 |
| | AT3G02610 | putative/stearoyl-ACP | | | |
| | | desaturase, putative; | | | |
| | | [AT3G02610]acyl-desaturase | | | |
| 254644_at | AT4G18510 | CLE2; receptor binding | 4 | 0 | 0.42 |
| 253767_at | AT4G28520 | CRU3; nutrient reservoir | 3 | 0 | 0.41 |
| 249082_at | AT5G44120 | CRA1; nutrient reservoir | 0 | 0 | 0.11 |

*The sequences of the genes identified above can be identified by reference to either or both of the Affy ID number of Gene number. The sequences associated with these identification numbers are incorporated herein by reference.

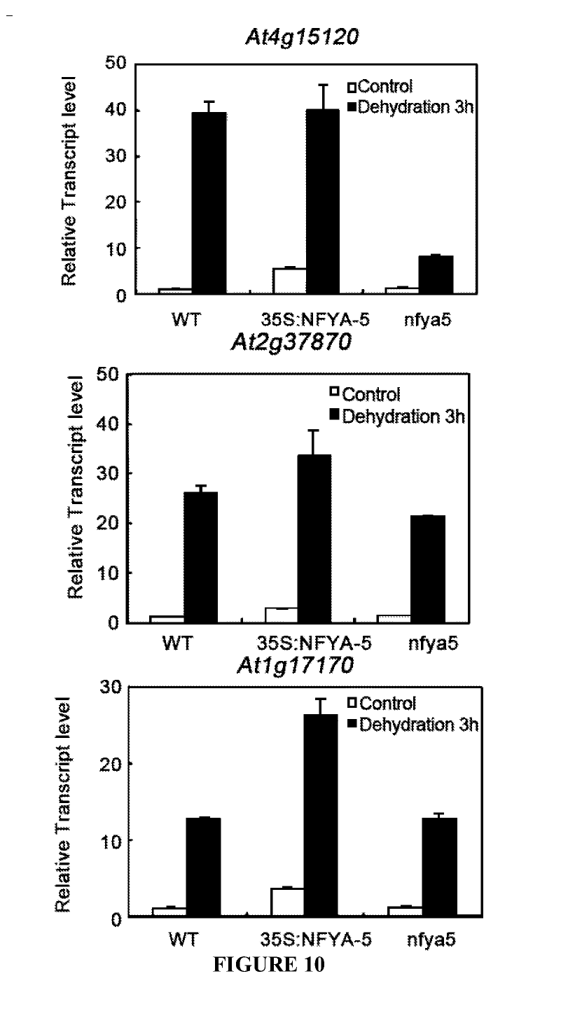

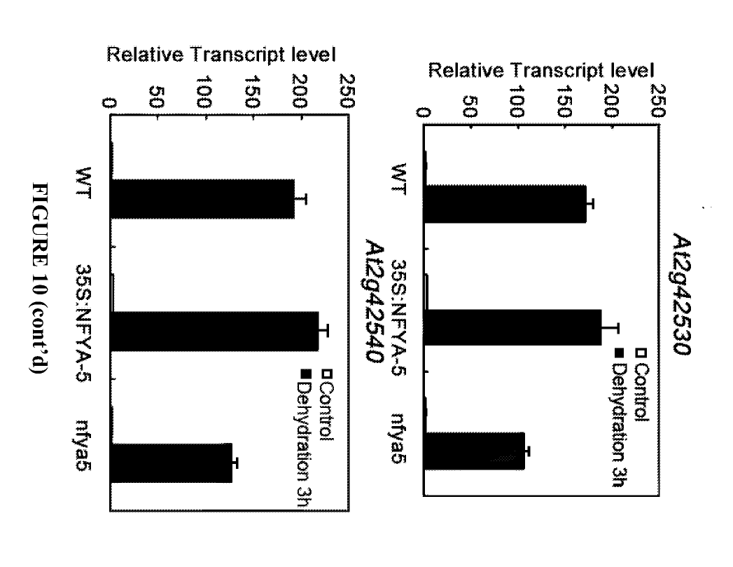

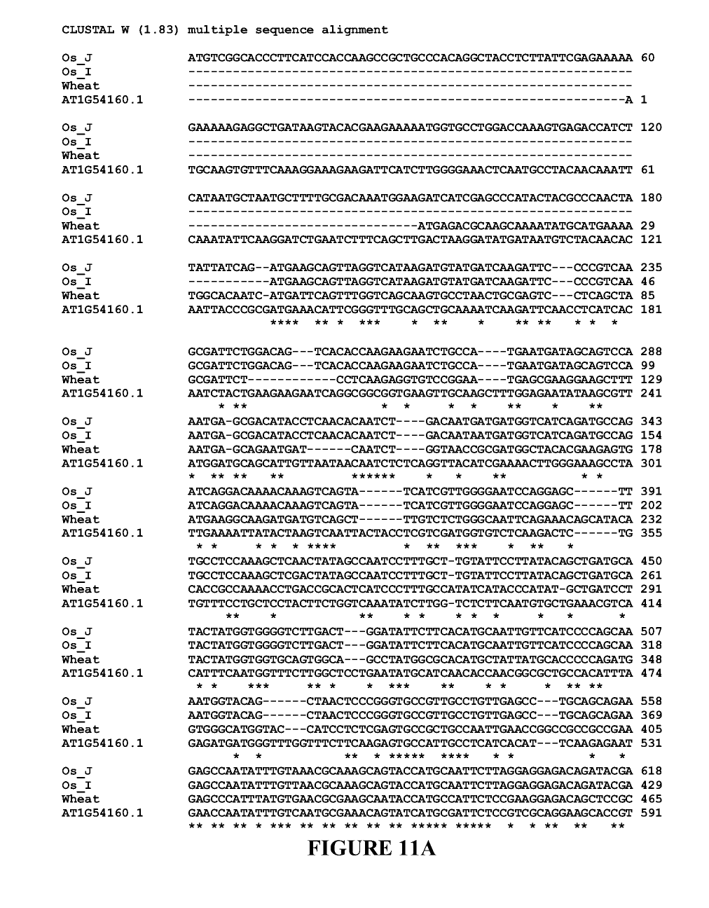

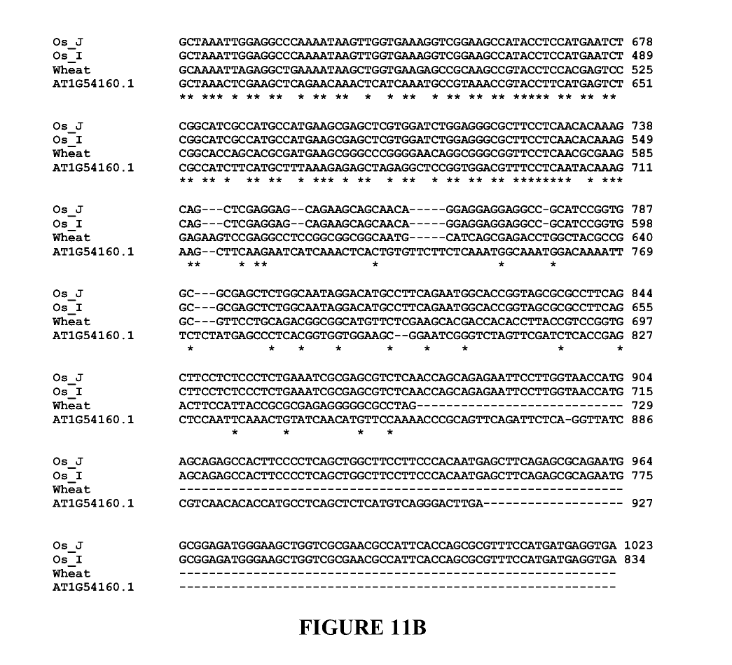

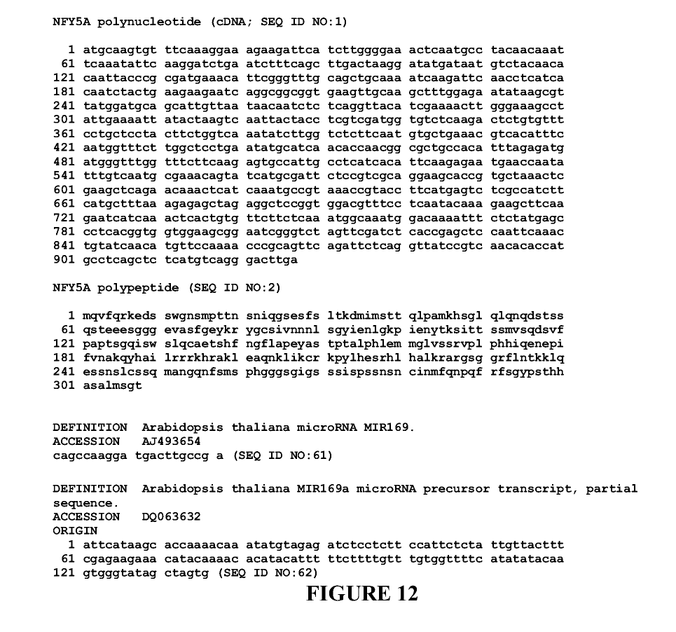

The microarray results were confirmed by real-time RT-PCR. In agreement with the microarray data, the real time RT-PCR assay showed that At4g15210 (cytosolic beta-amylase), At2g37870 (protease inhibitor), At1g17170 (glutathione transferase), At2g42530 (cold-responsive protein) and At2g42540 (COR15A) were expressed at higher levels in 35S:NFYA5 plants under normal conditions (FIG. 10), suggesting constitutive expression of stress responsive genes in 35S:NFYA5. Under dehydration conditions, these genes were strongly induced in the wild type and 35S:NFYA5; however, dehydration-induced accumulation of the majority of these genes was substantially reduced in nfya5, suggesting that optimal dehydration stress induction of many of these genes requires NFYA5.

The foregoing examples are illustrative only and are not meant to limit the disclosure.

1. An isolated polynucleotide comprising:
an NFY5A polynucleotide, homolog or otholog thereof operably linked to a heterologous promoter.

2. The isolated polynucleotide of claim 1, wherein the NFY5A comprises at least 50%, 60%, 70%, 80%, 90%, 95%, 98% or 99% identity to a polynucleotide selected from the group consisting of SEQ ID NO:1, 3, 5, or 7.

3. The isolated polynucleotide of claim 1, wherein the NFY5A comprises SEQ ID NO:1, 3, 5, or 7.

4. The isolated polynucleotide of claim 1, wherein the NFY5A polynucleotide encodes a polypeptide comprising SEQ ID NO: 2, 4, 6, or 8.

5. The isolated polynucleotide of claim 1, wherein the NFY5A polynucleotide encodes a polypeptide having at least 80% identity to SEQ ID NO: 2, 4, 6, or 8 wherein the polypeptide modified stomata openings in a plant cell.

6. The isolated polynucleotide of claim 1, wherein the heterologous promoter comprises a constitutive promoter.

7. The isolated polynucleotide of claim 1, wherein the heterologous promoter comprises an inducible promoter.

8. The polynucleotide of claim 1, contained in an expression vector.

9. The polynucleotide of claim 6, wherein the constitutive promoter is selected from the group consisting of a cauliflower mosaic virus 35S or 19S promoter, a plant ACT2 promoter and a plant ubiquitin promoter.

10. The polynucleotide of claim 6, wherein the constitutive promoter is an Actin2 promoter derived from Arabidopsis thaliana.

11. The polynucleotide of claim 7, wherein the inducible promoter comprises a light inducible promoter.

12. The polynucleotide of claim 11, wherein said light inducible promoter is a SRS1 promoter of Glycine max.

13. A plant cell transformed with the polynucleotide of claim 1.

14. A transgenic plant transformed with a Polynucleotide of claim 1, wherein the plant expresses a NFY5A polynucleotide at a level higher than that of a wild-type plant and the transgenic plant has improved drought tolerance.

15. A transgenic plant that lacks the 3′UTR of a NFY5A gene, homolog or ortholog thereof and which has improved drought tolerance compared to a wild-type plant.

16. A transgenic plant that comprising a reduced expression of an miR169a or c polynucleotide and comprises improved drought tolerance compared to a wild-type plant.

17. The transgenic plant of claim 16, wherein the miR169 is at least 98% or 99% identical to SEQ ID NO:61.

18. A transgenic plant comprising a knockout of an miR169a or c polynucleotide and having improved drought tolerance compared to a wild-type plant.

19. A plant part or cell obtained from the transgenic plant of of claims 14-18.

20. A method of using the polynucleotide of claim 1 to produce a plant which is drought resistant, comprising introducing the polynucleotide into a plant cell or into plant tissue, selecting for the presence of the polynucleotide molecule to produce a transgenic plant cell or transgenic plant tissue, and regenerating a plant from the transgenic plant cell or transgenic plant tissue, whereby a drought resistant plant is generated.

21. A method for identifying agents useful for modifying drought resistance in a plant comprising:
contacting a plant that contains an NFY5A gene with an agent;
measuring a change in expression of the gene or the amount of mRNA transcript present in the cell;
wherein an increase in expression or transcript levels compared to a control is indicative of an agent that improves drought tolerance.

22. A method for identifying agents useful for modifying drought tolerance in a plant comprising:
contacting a plant with an agent, wherein the plant comprises a sequence of TX(C/A)TTXGX(C/A)CAXT (SEQ ID NO:60) operably linked to a reporter gene;
measuring a change in expression of the reporter gene;
wherein a change in the expression or the reporter gene compared to a control is indicative of an agent that modifies drought tolerance.

23. A method of increasing drought tolerance in a plant comprising contact a plant with an agent the increases the expression of (1) an NFY5A gene, homolog or ortholog thereof and/or (2) a gene listed in Table 2.
